(19) United States
(12) Patent Application Publication
(10) Pub. No.: US 2026/0366978 A1
(43) Pub. Date: Nov. 19, 2026

(54) METHOD AND DEVICE FOR SINGLE PARTICLE BIOANALYSIS

(71) Applicant: The University of Tokyo, Tokyo (JP)

(72) Inventors: Ken ISHII, Tokyo (JP); Koji KOBIYAMA, Tokyo (JP); Hideo NEGISHI, Tokyo (JP); Burcu TEMIZOZ, Tokyo (JP); Tomoya HAYASHI, Tokyo (JP)

(73) Assignee: The University of Tokyo, Tokyo (JP)

(21) Appl. No.: 19/134,919

(22) PCT Filed: Nov. 30, 2023
(86) PCT No.: PCT/JP2023/042988

§ 371 Date: Apr. 6, 2026

(30) Foreign Application Priority Data
Dec. 2, 2022 (JP) 2022-193517

(51) Int. Cl. G01N 15/0227

(52) U.S. Cl. CPC G01N 15/0227

(57) ABSTRACT

The invention provides an analysis method for extracellular microparticles including, for each extracellular microparticle in a population of extracellular microparticles, generating a feature vector by detecting light scattered from incident light and a plurality of luminescent emissions having different peak wavelengths generated from components of the extracellular microparticle or labeling substances bound thereto; creating a first scatter diagram by mapping at least a portion of the extracellular microparticles contained in the population of extracellular microparticles on the basis of the similarity of representative vectors composed of part or all of the elements of the feature vectors, and labeling each of the mapped extracellular microparticles on the basis of at least a plurality of the elements corresponding to the detected luminescent emissions among the elements of the representative vectors; and classifying the mapped extracellular microparticles into two or more clusters on the basis of the first scatter diagram.

### TECHNICAL FIELD

The present invention relates to an analysis method and an analysis device for single particle bioanalysis, and an analysis sample preparation method, and the like.

### BACKGROUND ART

Extracellular microparticles ranging from several tens of nanometers to several micrometers, which include bioactive molecules such as nucleic acids, are present in vivo, and their involvement in various diseases has been focused upon. For example, during influenza virus infection, in addition to virus particles, host cell-derived vesicles and nucleic acids such as neutrophil extracellular traps are released outside cells and involved in the pathogenesis. Therefore, the quantification of these extracellular microparticles may serve as a new index for evaluating the pathology of diseases and the effectiveness of vaccines and therapeutic agents. However, although analysis methods at the protein level and single-cell level have been established, effective techniques for analyzing extracellular microparticles at the single-particle level have not yet been established.

In recent years, flow cytometry has been focused upon for single-particle analysis of nanoparticles, including extracellular vesicles. Because flow cytometry is a tool developed for cell analysis, until now, it has been technically difficult to analyze nanoparticles 1/100 of the size of cells by flow cytometry. However, as the detection sensitivity of flow cytometers has improved, size measurement based on scattered light and one- or two-color analysis using fluorescently labeled antibodies or low-molecular-weight fluorescent reagents have been reported (Non-Patent Literature 1 to 4).

### CITATION LIST

### Non-Patent Literature

Non-Patent Literature 1: Brittain, G.C.IV. et al. A Novel Semiconductor-Based Flow Cytometer with Enhanced Light-Scatter Sensitivity for the Analysis of Biological Nanoparticles. Sci. Rep. 9, 16039 (2019).
Non-Patent Literature 2: Danielson, K. M. et al. Diurnal Variations of Circulating Extracellular Vesicles Measured by Nano Flow Cytometry. PLoS One 11, e0144678 (2016).
Non-Patent Literature 3: van der Vlist, E. J. et al. Fluorescent labeling of nano-sized vesicles released by cells and subsequent quantitative and qualitative analysis by high-resolution flow cytometry. Nat. Protoc. 7, 1311-1326 (2012).
Non-Patent Literature 4: Morales-Kastresana, A. et al. Labeling Extracellular Vesicles for Nanoscale Flow Cytometry. Sci. Rep. 7, 1878 (2017).

### SUMMARY OF INVENTION

### Technical Problem

However, in analysis techniques reported so far, only some information about microparticles, for example, information about specific marker proteins or constituent molecules, can be obtained.

Therefore, an object of the present invention is to provide a method, device and the like for evaluating characteristics of extracellular microparticles.

### Solution to Problem

An analysis method for extracellular microparticles according to one aspect of the present invention includes, for each extracellular microparticles in a population of extracellular microparticles, generating a feature vector corresponding to the extracellular microparticle by detecting light scattered from incident light and a plurality of luminescent emissions having different peak wavelengths, the luminescent emissions being generated from components of the extracellular microparticle or labeling substances bound to the components in response to the incident light; creating a first scatter diagram by mapping, in two or three dimensions, at least a portion of the extracellular microparticles contained in the population of extracellular microparticles on the basis of the similarity of representative vectors composed of part or all of the elements of the feature vectors, and labeling each of the mapped extracellular microparticles on the basis of at least a plurality of the elements corresponding to the detected luminescent emissions among the elements of the representative vectors; and classifying the mapped extracellular microparticles into two or more clusters on the basis of the first scatter diagram.

According to this analysis method, since a population of extracellular microparticles is mapped in two or three dimensions on the basis of the similarity of vectors generated based on the scattered light and a plurality of the luminescent emissions detected when irradiating the extracellular microparticles with light, it is possible to visualize the distribution of similarity of the population of extracellular microparticles on the basis of a plurality of characteristics such as the size and components. In addition, by labeling each of the mapped extracellular microparticles on the basis of at least a plurality of the elements corresponding to the luminescent emissions among the elements of the representative vectors, information regarding characteristics of the components of the extracellular microparticles can be additionally assigned to the extracellular microparticles for which the distribution of similarity was visualized. In addition, using the first scatter diagram thus obtained, the extracellular microparticles can be classified into two or more clusters. In this manner, according to the analysis method according to this aspect, characteristics of the population of extracellular microparticles can be subjected to cluster analysis at the single-particle level, and the characteristics of the population of extracellular microparticles can be comprehensively clarified.

Preferably, the analysis method according to the above aspect further includes creating a second scatter diagram by mapping at least a portion of the extracellular microparticles contained in the population of extracellular microparticles in the same manner as the first scatter diagram, and visualizing one of a plurality of the elements corresponding to the detected luminescent emissions among the elements of the representative vectors in each of the mapped extracellular microparticles. When the second scatter diagram is created as described above, it is possible to identify, among the extracellular microparticles mapped on the first scatter diagram, for example, extracellular microparticles containing a large amount or a negligible amount of a predetermined component and the cluster to which the extracellular microparticles belong.

In addition, in this aspect, preferably, the method further includes displaying the first and second scatter diagrams; and identifying characteristics of at least one of the clusters on the basis of the first and second scatter diagrams displayed. According to this aspect, a user can compare the first scatter diagram with the second scatter diagram, can confirm the content of a predetermined component in extracellular microparticles contained in at least one of clusters in the first scatter diagram, and thus can identify in more detail what type of extracellular microparticles the cluster contains. Here, displaying of the first and second scatter diagrams may be performed by an analysis device for extracellular microparticles and/or an information processing unit provided therein, and identification of characteristics may be performed by the analysis device for extracellular microparticles and/or the information processing unit provided therein, and/or a user.

In the analysis method according to the above aspect, it is preferable that, in creating the first or second scatter diagram, extracellular microparticles in which all of the elements corresponding to the detected luminescent emissions among the elements of the representative vectors are equal to or less than a threshold are excluded from mapping. According to this aspect, it is possible to exclude from analysis extracellular microparticles having low contents of all components of the extracellular microparticles to be analyzed, and it is possible to further improve the accuracy of creating the first scatter diagram and the cluster analysis.

In the analysis method according to the above aspect, the population of extracellular microparticles is preferably a population of extracellular microparticles in which at least three types selected from the group consisting of nucleic acids, proteins, lipids, and sugar chains are stained, and more preferably a population of extracellular microparticles in which all of the nucleic acids, proteins, lipids, and sugar chains are stained. According to this aspect, it is possible to further improve the accuracy of creating the first scatter diagram and the cluster analysis.

An analysis method for extracellular microparticles according to another aspect of the present invention includes, for each extracellular microparticle in a population of extracellular microparticles, generating a feature vector corresponding to the extracellular microparticle by detecting light scattered from incident light and a plurality of luminescent emissions having different peak wavelengths, the luminescent emissions being generated from components of the extracellular microparticle or labeling substances bound to the components in response to the incident light; and classifying the population of extracellular microparticles into two or more clusters on the basis of the similarity of representative vectors composed of part or all of the elements of the feature vectors.

According to this analysis method, since the population of extracellular microparticles is classified into two or more clusters on the basis of the similarity of vectors generated based on the scattered light and a plurality of the luminescent emissions detected when irradiating the extracellular microparticles with light, it is possible to classify a plurality of extracellular microparticles into two or more clusters on the basis of a plurality of characteristics such as the size and components. In this manner, according to the analysis method according to this aspect, characteristics of the population of extracellular microparticles can be subjected to cluster analysis at the single-particle level, and the characteristics of the population of extracellular microparticles can be comprehensively clarified.

An analysis method for extracellular microparticles according to another aspect of the present invention includes irradiating an extracellular microparticle with incident light, and detecting light scattered from the incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle; and analyzing characteristics of extracellular microparticle on the basis of analysis data obtained by analyzing a population of extracellular microparticles by the analysis method according to any one of the above aspects and the acquired feature vectors.

This analysis method is a method of analyzing extracellular microparticles based on analysis data obtained by analyzing a population of extracellular microparticles by the analysis method according to any one of the above aspects. Therefore, since an extracellular microparticle can be analyzed using the analysis results of a separately analyzed population of extracellular microparticles, the extracellular microparticle can be analyzed more easily.

A separation method for extracellular microparticles according to another aspect of the present invention includes, for an extracellular microparticle in a population of extracellular microparticles, detecting light scattered from incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle; determining whether the extracellular microparticle has a predetermined characteristic on the basis of analysis data obtained by analyzing a population of extracellular microparticles by the analysis method according to any one of the above aspects and the acquired feature vector; and separating, when determination is made that the extracellular microparticle has the predetermined characteristic, the extracellular microparticle from the population of extracellular microparticles.

This separation method is a method of separating an extracellular microparticle having a predetermined characteristic from a population of extracellular microparticles on the basis of analysis data obtained by analyzing a population of extracellular microparticles by the analysis method according to any one of the above aspects. According to this method, it is possible to separate only a extracellular microparticle having a specific characteristic from the population of extracellular microparticles and further, for example, analyze the extracellular microparticles.

An analysis device for extracellular microparticles according to another aspect of the present invention includes a detection unit configured to detect, for each extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light; and an information processing unit including a feature vector generating means for generating a feature vector corresponding to the extracellular microparticle in the population of extracellular microparticles on the basis of the detected scattered light and plurality of luminescent emissions, a scatter diagram creating means for creating a first scatter diagram by mapping, in two or three dimensions, at least a portion of the extracellular microparticles contained in the population of extracellular microparticles on the basis of the similarity of representative vectors composed of part or all of the elements of the acquired feature vectors, and labeling each of the mapped extracellular microparticles on the basis of at least a plurality of the elements corresponding to the detected luminescent emissions among the elements of the representative vectors, and a cluster generating means for generating two or more clusters, to which the extracellular microparticles belong, by assigning cluster information to each of the extracellular microparticles mapped on the first scatter diagram.

According to this analysis device, the analysis method according to the above aspect can be performed. That is, according to the analysis device according to this aspect, characteristics of the population of extracellular microparticles can be subjected to cluster analysis at the single-particle level, and the characteristics of the population of extracellular microparticles can be comprehensively clarified.

An analysis device for extracellular microparticles according to another aspect of the present invention includes a detection unit configured to irradiate an extracellular microparticle with incident light and detect light scattered from incident light and a plurality of luminescent emissions having different peak wavelengths, the luminescent emissions being generated from components of the extracellular microparticle or labeling substances bound to the components in response to the incident light; and an information processing unit including a feature vector generating means for generating a feature vector corresponding to the extracellular microparticles on the basis of the detected scattered light and the plurality of luminescent emissions and a characteristic analysis means for analyzing characteristics of the extracellular microparticle on the basis of analysis data obtained by analyzing a population of extracellular microparticles by the analysis method according to any one of the above aspects and the acquired feature vector.

According to this analysis device, it is possible to analyze the extracellular microparticles on the basis of analysis data obtained by analyzing a population of extracellular microparticles by the analysis method according to any one of the above aspects. Therefore, since the extracellular microparticle can be analyzed using the analysis results of a separately analyzed population of extracellular microparticles, the extracellular microparticle can be analyzed more easily.

A separation device for extracellular microparticles according to another aspect of the present invention includes a detection unit configured to detect, for an extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light; an information processing unit including a feature vector generating means for generating a feature vector corresponding to the extracellular microparticle on the basis of the detected scattered light and plurality of luminescent emissions and a determination means for determining whether the extracellular microparticle has a predetermined characteristic on the basis of analysis data obtained by analyzing a population of extracellular microparticles by the analysis method according to any one of the above aspects and the acquired feature vectors; and a separation unit configured to separate, when determination is made that the extracellular microparticle has the predetermined characteristic, the extracellular microparticle from the population of extracellular microparticles.

According to this separation device, it is possible to separate an extracellular microparticle having a predetermined characteristic from a population of extracellular microparticles on the basis of analysis data obtained by analyzing a population of extracellular microparticles by the analysis method according to any one of the above aspects. According to this separation device, it is possible to separate only an extracellular microparticle having a specific characteristic from the population of extracellular microparticles and further analyze the extracellular microparticle.

An preparation method for sample for extracellular microparticle analysis according to another aspect of the present invention includes labeling a sample containing a population of extracellular microparticles with a plurality of labeling substances that bind to components of the extracellular microparticles. This preparation method is an example of a method of preparing a sample containing a population of extracellular microparticles for use in the analysis method according to any one of the above aspects.

In this preparation method, it is preferable that the plurality of labeling substances include at least three types selected from the group consisting of a nucleic acid staining reagent, a protein staining reagent, a lipid staining reagent, and a sugar chain staining reagent. In addition, it is also preferable that the plurality of labeling substances include a compound having a molecular weight of 20,000 or less. According to these aspects, it is possible to provide an analysis sample that is more suitable for the analysis method according to the above aspect.

### Advantageous Effect of Invention

According to the present invention, it is possible to provide a method, device and the like for evaluating characteristics of extracellular microparticles.

### BRIEF DESCRIPTION OF DRAWINGS

FIG. 1 is a flowchart showing an example of an extracellular microparticle analysis method according to the present embodiment.

FIG. 2 is a flowchart showing an example of information processing in the extracellular microparticle analysis method according to the present embodiment.

FIG. 3 shows an example of a configuration of an extracellular microparticle analysis device according to the present embodiment.

FIG. 4 is a block diagram showing an example of a functional configuration of an information processing unit in the extracellular microparticle analysis device according to the present embodiment.

FIG. 5 is a block diagram showing an example of a physical configuration of the information processing unit in the extracellular microparticle analysis device according to the present embodiment.

FIG. 6 shows detection and sorting of extracellular microparticles using optimized forward scattering in BD Influx. a, b: an overview of optical system of BD Influx (high-resolution cell sorter) (a) and BD Aria III (conventional cell sorter) (b). c to f: a mixture of 100, 200, 500, and 1,000 nm fluorescent beads was analyzed by FSC threshold triggering with BD Influx (c) or BD Aria III (d). BD Aria III failed to detect 100, 200, and 500 nm beads due to saturation of noise signals. Beads detected with BD Influx (1000, 500, 200, and 100 nm) or BD Aria III (1,000 nm) were sorted and reanalyzed with BD Influx (e, f). Pie charts show the proportions of the beads and the purity of the sorted beads (center of the chart).

FIG. 7 shows multiparametric analysis of extracellular microparticles contained in BALF during influenza virus infection. a to c: BALF was collected from mice not infected with influenza virus or mice 4 days after infection. (b) cells and extracellular microparticles in BALF were measured with BD Influx without droplet formation. In order to analyze the morphology of extracellular microparticles, BALF was centrifuged at 400×g, the extracellular microparticles in the supernatant were observed under a transmission electron microscope (TEM) after negative staining. (c) extracellular microparticles in BALF were measured with BD Influx after staining with SYBR gold, Hoechst33258, CellMask deep red, BV510 annexin V, CF05M WGA, CF568 PNA, Live/Dead Near-IR, and R-PE-CF647T anti-HA antibodies. d: all extracellular microparticles detected in b were analyzed by t-SNE. Each stained particle was visualized by the color indicated in the drawing. The intensity of each channel is shown as a heat map. e: an overview of a gating strategy for excluding noise signals and unstained particles from subsequent t-SNE analysis. The stained particle of each color was gated, and extracted by setting an “or” gate. f: the results of analyzing the stained particle by t-SNE were shown in the same manner as in d.

FIG. 8 shows evaluation of the severity of virus infection by multiparametric analysis of both cells and extracellular microparticles in BALF. a, b: BALF from mice infected with PR8 virus with an LD₅₀ of 0.1, 1, or 10 or from uninfected mice was stained with SYBR gold, Hoechst33258, BV510 annexin V, CF568 PNA, and HA, CD11c, siglec-F, CD3e, Ly6G and Ly6C antibodies. Cells and stained particles were gated and analyzed by t-SNE. (a) representative data of t-SNE analysis of cells of respective groups (top) and stained particles (bottom). Respective cell groups or stained particles were visualized by the color indicated in the drawing. (b) The numbers of respective particles and cells in the 2-minute measurement are shown.

FIG. 9 shows high-performance purification of stained particles by flow cytometric sorting. a: BALF from mice infected with PR8 virus with an LD₅₀ of 10 was stained with SYBR gold, Hoechst33258, CellMask deep red, CF405M WGA, LiveDead Near-IR, and RPE-CF647T anti-HA antibodies. According to the gating strategy shown in c, HA-positive, SYBR gold-positive, WGA-positive, PNA-positive, Annexin V-positive, and CellMask-positive particles were sorted. Reanalysis of the sorted particles was performed after fractions sorted with SYBR gold were stained. BALF was also processed by differential centrifugation (DC) or size exclusion chromatography (SEC), and unpurified samples were measured in the same manner. Stained particles detected in unpurified samples, and samples purified by DC or SEC were gated, extracted by setting an “or” gate, and analyzed by t-SNE. Target particles in the sorted fractions were also gated and subjected to t-SNE analysis after setting an “or” gate. Pie charts show the proportions of the groups gated in the t-SNE plot. b: total RNA was extracted from the sorted fractions with an equal volume. The amount of PR8 segment 5 viral RNA was quantified by RT-qPCR. c: a gating strategy used for sorting the groups.

FIG. 10 shows sorting of a mixture of 100, 200, 500, and 1,000 nm fluorescent beads using a BD FACSDiscover S8.

FIG. 11 shows multiparametric analysis of extracellular microparticles contained in BALF during influenza virus infection using the BD FACSDiscover S8.

FIG. 12 shows high-performance purification of stained particles using the BD FACSDiscover S8.

### DESCRIPTION OF EMBODIMENTS

Hereinafter, a form for implementing the present invention (hereinafter referred to as “the present embodiment”) will be described in detail with reference to the drawings, but the present invention is not limited thereto, and can be variously modified without departing from the spirit and scope thereof. In the following description of the drawings, the same or similar components will be denoted with the same or similar reference numerals. The drawings are schematic, and do not necessarily match actual dimensions, ratios, and the like. The drawings may also include components having different dimensional relationships and ratios with each other.

### [Analysis Method]

An analysis method for extracellular microparticles according to the present embodiment includes, for each extracellular microparticle in a population of extracellular microparticles, generating a feature vector corresponding to the extracellular microparticle by detecting light scattered from incident light and a plurality of luminescent emissions having different peak wavelengths generated from components of the extracellular microparticle or labeling substances bound to the components in response to the incident light; creating a first scatter diagram by mapping at least a portion of the extracellular microparticles contained in the population of extracellular microparticles in two or three dimensions based on the similarity of representative vectors composed of part or all of the elements of the feature vectors, and labeling each of the mapped extracellular microparticles based on at least a plurality of the components corresponding to the detected luminescent emissions among the elements of the representative vectors; and classifying the mapped extracellular microparticles into two or more clusters based on the first scatter diagram.

In the analysis method of the present embodiment, a plurality of characteristics such as the size and components of each extracellular microparticle are acquired at the single-particle level, the obtained data is analyzed by information processing, and thus the extracellular microparticles are subjected to cluster analysis based on a plurality of types of information. This makes it possible to identify extracellular microparticles that serve as biomarkers for disease onset, disease cause, and/or prognosis prediction from various extracellular microparticles contained in biological samples. In addition, it is also possible to evaluate characteristics of environmental microparticles or various nanoparticles contained in pharmaceuticals and the like at the single-particle level.

Although a method of staining cells with multiple colors and analyzing the cells based on a plurality of types of information is known, the analysis method of the present embodiment is an analysis technique that is completely different from such multicolor cell analysis. That is, compared with cells, extracellular microparticles have a surface area of about 1/100 to 1,000,000, and a volume of about 1/1,000 to 1,000,000,000, and due to the bulkiness of the staining reagent, it is difficult to stain extracellular microparticles with multiple colors using the same method as for the cells. Therefore, in existing prior art, including the above Non-Patent Literature, extracellular microparticles have not been subjected to multicolor analysis, and only specific marker proteins have been analyzed by staining one or two components. That is, there have been no attempts to comprehensively analyze extracellular microparticles at the single-particle level based on a plurality of types of information. According to the analysis method of the present embodiment, the extracellular microparticles can be comprehensively analyzed at the single-particle level by visualizing a plurality of characteristics of extracellular microparticles.

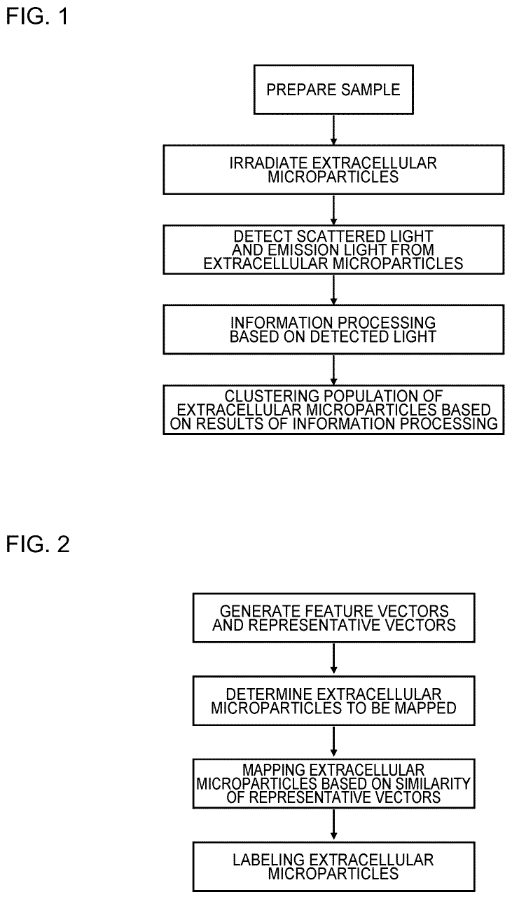

FIG. 1 is a flowchart showing an example of an analysis method according to the present embodiment. Hereinafter, the analysis method of the present embodiment will be appropriately described with reference to the drawings. Here, the analysis method shown in FIG. 1 and the following description is an example of the analysis method according to the present embodiment, but descriptions thereof are not intended to limit the present invention. For example, the analysis method of the present embodiment may not include a step of preparing a sample. In this case, for example, a sample containing a population of extracellular microparticles whose components have already been labeled may be obtained and analyzed.

### (Preparation of Sample)

In FIG. 1, first, a sample to be used in the analysis method of the present embodiment is prepared. The prepared sample contains a population of extracellular microparticles. In the analysis sample, the extracellular microparticles contain a component that emits light in response to light emission, or if they do not contain such a component, they contain a component to which a labeling substance that emits light in response to light emission binds. Therefore, the preparation of the sample preferably includes binding a labeling substance to a component of the extracellular microparticles. Examples of components that emit light in response to light emission include components that generate autofluorescence such as melanin.

In this specification, the term “extracellular microparticles” refers to particles having a size of several tens of nanometers to several micrometers that are released outside cells, including extracellular vesicles such as exosomes, microvesicles and apoptotic bodies, as well as extracellular particles such as protein-nucleic acid complexes. The size of the extracellular microparticles may be, for example, 1.0 nm or more and 50 μm or less, 10 nm or more and 10 μm or less, or 50 nm or more and 5.0 μm or less.

In this specification, the term “labeling substance” refers to a substance that specifically binds to a specific component of extracellular microparticles and can be optically detected by light emission. Here, the mode in which a labeling substance binds to a component is not particularly limited, and examples thereof include covalent bonds, bonds due to hydrophobic interactions, hydrogen bonds, and ionic bonds. The labeling substance may be a substance that generates scattered light in response to light emission, a substance that emits light, and particularly a substance that generates fluorescence. The labeling substance may be, for example, a staining reagent for a component of extracellular microparticles.

In this specification, the term “staining” means that a molecule that emits light (typically emits fluorescence light) in response to light emission is specifically chemically or physically bound to a specific component of extracellular microparticles and specifically chemically or physically binding a molecule that emits light (typically emits fluorescence light) in response to light emission to a specific component of extracellular microparticles. In addition, the term “staining reagent” refers to a substance for specifically staining a specific component of extracellular microparticles, that is, a substance that specifically binds to a specific component of extracellular microparticles and emits light (typically emits fluorescence light) in response to light emission.

The components of extracellular microparticles to be labeled with a labeling substance preferably include at least one of nucleic acids, proteins, lipids and sugar chains, more preferably include at least three types of nucleic acids, proteins, lipids and sugar chains, and still more preferably include all of nucleic acids, proteins, lipids and sugar chains. When these components are labeled and detected, the population of extracellular microparticles can be subjected to cluster analysis with higher accuracy. In the analysis sample, constituent molecules other than nucleic acids, proteins, lipids and sugar chains may be labeled with a labeling substance.

In the analysis sample, preferably, three or more types of components of the extracellular microparticles are labeled with a labeling substance, and more preferably, four or more types of components are labeled with a labeling substance. In addition, the components of the extracellular microparticles may be labeled with preferably three or more types of labeling substances, more preferably four or more types of labeling substances, still more preferably five or more types of labeling substances, yet more preferably six or more types of labeling substances, even more preferably seven or more types of labeling substances, and particularly preferably eight or more types of labeling substances. The upper limit of the number of types of labeling substances contained in the population of extracellular microparticles is not particularly limited, and may be for example, 40 types, 30 types, 20 types, 16 types, 15 types, 12 types or 10 types.

The analysis sample preferably contains a population of extracellular microparticles in which at least three types selected from the group consisting of nucleic acids, proteins, lipids and sugar chains are stained. In this case, in the population of extracellular microparticles, molecules other than nucleic acids, proteins, lipids and sugar chains may be stained. In order to improve the accuracy of cluster analysis to be described below, it is preferable that all of the nucleic acids, proteins, lipids and sugar chains in the population of extracellular microparticles be stained.

In this case, in the population of extracellular microparticles, each of the nucleic acids, proteins, lipids and sugar chains may be stained with one type, or one or more types of staining reagents. Each of the nucleic acids, proteins, lipids and sugar chains may be stained with one or more and 10 or less types, one or more and eight or less types, one or more and five or less types, two or more and 10 or less types, two or more and eight or less types, or two or more and five or less types of staining reagents.

In addition, the population of extracellular microparticles is preferably stained with three or more types of staining reagents, more preferably four or more types of staining reagents, still more preferably five or more types of staining reagents, yet more preferably six or more types of staining reagents, even more preferably seven or more types of staining reagents, and particularly preferably eight or more types of staining reagents. The upper limit of the number of types of staining reagents contained in the population of extracellular microparticles is not particularly limited, and may be, for example, 40, 30, 20, 16, 15, 12 or 10.

As described above, the volume and surface area of the extracellular microparticle are extremely small compared to the cells. Therefore, when the extracellular microparticles are labeled with a plurality of labeling substances, the labeling substance or staining reagent in the extracellular microparticles preferably contains a substance other than antibodies, more preferably contains a compound having a molecular weight of 20,000 or less, and still more preferably contains a low-molecular-weight compound. For example, the extracellular microparticles may contain, as a nucleic acid and/or lipid labeling substance or staining reagent, a compound having a molecular weight of 20,000 or less or a low-molecular-weight compound. The molecular weight of the low-molecular-weight compound may be, for example, 500 or less or 400 or less, and may be 100 or more or 200 or more. The labeling substance or staining reagent in the extracellular microparticle may contain a medium-molecular-weight compound having a molecular weight of 500 or more and 20,000 or less.

The nucleic acid staining reagent is not particularly limited as long as it is a substance that can stain nucleic acids, and examples thereof include those shown in the following table. The nucleic acids to be stained may be selected depending on the sample and the purpose of analysis. As the nucleic acid staining reagent, a staining reagent with a binding mode of “Bis-intercalator” and a staining reagent with a binding mode of “Minor groove binder” are preferable, and among these, a staining reagent with a binding mode of “Bis-intercalator” and a staining reagent with a binding mode of “Minor groove binder” in the following table are more preferable.

TABLE 1

| | | | Excitation | |
|---|---|---|---|---|
| | | | wavelength/ | |
| Binding | | | emission light | Storage |
| mode | Staining reagent | Manufacturer | wavelength (nm) | solution |
| Intercalator | PI (Propidium iodide) | Sigma Aldrich | 493/636 | 1 mg/mL in H₂O |
| | Acridine orange | Biotium | 502/526 | 1 mg/mL in H₂O |
| | | | (DNA), | |
| | | | 460/650 | |
| | | | (RNA) | |
| | 7-AAD (7- | eBioscience | 548/648 | In PBS |
| | aminoactinomycin D) | | | |
| | Quinacrine mustard | Cayman | 425/480 | 10 mg/mL in |
| | (hydrochloride) | Chemical | | DMSO |
| | ACMA (9-Amino-6- | Cayman | 411/475 | 1 mg/mL in |
| | chloro-2- | Chemical | | DMSO |
| | methoxyacridine) | | | |
| Bis- | SYBR gold | Invitrogen | 495/537 | 10000 x in |
| intercalator | | | | DMSO |
| | SYBR green I | Invitrogen | 494/521 | 10000 x in |
| | | | | DMSO |
| | SYBR green II | Invitrogen | 497/520 | 10000 x in |
| | | | | DMSO |
| | YOYO-1 iodide | Invitrogen | 491/509 | 1 mM in DMSO |
| | YO-PRO-1 iodide | Invitrogen | 491/509 | 1 mM in DMSO |
| | Thiazole orange | Biotium | 512/533 | 10 mM in DMSO |
| Minor | DAPI | Biolegend | 360/460 | 1 mg/ml in H₂O |
| groove | | | (DNA), | |
| binder | | | 360/500 | |
| | | | (RNA) | |
| | Hoechst 33258 | FujiFilm | 350/461 | 1 mg/mL in H₂O |
| | Hoechst 33342 | FujiFilm | 352/461 | 1 mg/mL in H₂O |
| Others | DRAQ5 | Invitrogen | 488-647/633- | 5 mM in |
| | | | 710 | aqueous buffer |
| | Red dot 1 Far-red | Biotium | 662/694 | 200 x in H₂O |
| | SYTO RNAselect | Invitrogen | 490/530 | 5 mM in DMSO |
| | Nuclear Violet LCS1 | AAT Bioquet | 401/459 | 5 mM in DMSO |
| | Nuclear Blue DCS1 | Cayman | 350/461 | 5 mM in DMSO |
| | | Chemical | | |
| | DMAO | Biotium | 496/528 | 2 mM in DMSO |

TABLE 2

| | | | Excitation | |
|---|---|---|---|---|
| | | | wavelength/ | |
| Binding | | | emission light | Storage |
| mode | Staining reagent | Manufacturer | wavelength (nm) | solution |
| Others | SYTO 17 | Invitrogen | 621/634 | 5 mM in DMSO |
| | SYTO 40 | Invitrogen | 420/441 | 5 mM in DMSO |
| | SYTO 41 | Invitrogen | 430/454 | 5 mM in DMSO |
| | SYTO 42 | Invitrogen | 433/460 | 5 mM in DMSO |
| | SYTO 45 | Invitrogen | 455/484 | 5 mM in DMSO |
| | SYTO 59 | Invitrogen | 622/645 | 5 mM in DMSO |
| | SYTO 60 | Invitrogen | 652/678 | 5 mM in DMSO |
| | SYTO 61 | Invitrogen | 628/645 | 5 mM in DMSO |
| | SYTO 62 | Invitrogen | 652/676 | 5 mM in DMSO |
| | SYTO 64 | Invitrogen | 599/619 | 5 mM in DMSO |
| | Bacto View Live | Biotium | 500/520 | 500 X in DMSO |
| | Green | | | |
| | HCS NuclearMask | Invitrogen | 350/461 | 2000 X |
| | Blue stain | | | |
| | Vybrant DyeCycle | Invitrogen | 369/437 | 5 mM in DMSO |
| | Violet stain | | | |
| | SYTOX Blue | Invitrogen | 444/480 | 1 mM in DMSO |
| | SYTOX Green | Invitrogen | 504/523 | 30 mM in DMSO |
| | SYTOX Orange | Invitrogen | 547/570 | 250 mM in |
| | | | | DMSO |
| | SYTOX Red | Invitrogen | 640/658 | 5 mM in DMSO |
| | LDS751 | Cayman | 543/712 | 10 mM in DMSO |
| | | Chemical | | |
| | Hydroxystibamidine | Cayman | 323/450, 600 | 4% in H₂O |
| | | Chemical | (DNA), | |
| | | | 323/450 | |
| | | | (RNA) | |
| | Hexidium Iodide | AAT Bioquet | 518/600 | 10 mg/mL in |
| | | | | DMSO |
| | 1,6-diphenyl-1,3,5- | Biotium | 350/420 | 10 mg/mL in |
| | hexatriene | | | DMSO |
| | Chromomycin A3 | Cayman | 445/575 | 20 mg/mL in |
| | | Chemical | | DMSO |
| | Pyronin Y | Cayman | 540-550/560- | 10 mg/mL in |
| | | Chemical | 580 | DMSO |

The lipid staining reagent is not particularly limited as long as it is a substance that can stain the inside of lipids or lipid particles, and examples thereof include amphipathic intercalator-type staining reagents such as PKH67, DiA, DiB, Neuto-DiO, DiD, and CellMask (CellMask Green, CellMask Orange, and CellMask Deep Red); amine-reactive staining reagents that emit fluorescence in response to decomposition with esterase inside lipid bilayer membranes such as 5-carboxyfluorescein diacetate succinimidyl ester (CFSE), 6-carboxyfluorescein diacetate succinimidyl ester (CFSE), and cell trace violet (CTV); and substances in which proteins that bind to phosphatidylserine (PS) are fluorescently labeled such as Annexin V. The lipid to be stained may be selected depending on the sample and the purpose of analysis. The lipid staining reagent preferably includes an amphipathic intercalator-type staining reagent, more preferably includes CellMask, and still more preferably CellMask Deep Red.

The protein staining reagent is not particularly limited as long as it is a substance that can stain proteins, and examples thereof include fluorescently labeled antibodies and staining reagents for evaluating cell viability such as Live/Dead stain Near-IR and Live/Dead fixable dead cell stain. The proteins to be stained may be selected depending on the sample and the purpose of analysis.

The sugar chain staining reagent is not particularly limited as long as it is a substance that can stain sugar chains, and examples thereof include fluorescently labeled lectins such as CF405M WGA, and CF568 PNA. The sugar chains to be stained may be selected depending on the sample and the purpose of analysis.

When extracellular microparticles are stained, staining reagents having different peak wavelengths are selected from among the above staining reagents. Accordingly, light detected in the light detection described below can be matched with the staining reagent in one-to-one correspondence, and the population of extracellular microparticles can be subjected to cluster analysis with higher accuracy.

The method of preparing a sample for use in the analysis method of the present embodiment includes, for example, labeling a sample containing a population of extracellular microparticles with a plurality of labeling substances that bind to components of the extracellular microparticles, and preferably includes staining with at least three types selected from the group consisting of a nucleic acid staining reagent, a protein staining reagent, a lipid staining reagent, and a sugar chain staining reagent. The number of types of labeling substances or staining reagents used, and examples of labeling substances and staining reagents are as described above. For example, during sample preparation, it is preferable to stain a sample containing a population of extracellular microparticles with all of a nucleic acid staining reagent, a protein staining reagent, a lipid staining reagent, and a sugar chain staining reagent.

As the method of labeling a population of extracellular microparticles with a labeling substance or staining reagent, a known method may be appropriately used depending on the labeling substance or staining reagent used and an object to be labeled or stained. In addition, the components of the extracellular microparticles, for example, nucleic acids, proteins, lipids and sugar chains, may be stained simultaneously or stained sequentially. In addition, when two or more types of labeling substances or staining reagents are used for each of the components of the extracellular microparticles, for example, nucleic acids, proteins, lipids and sugar chains, the components may be labeled or stained simultaneously or sequentially with these two or more types of labeling substances or staining reagents. When the components are labeled or stained sequentially, the population of extracellular microparticles may be washed between labeling or staining with the first labeling substance or staining reagent and labeling or staining with the second labeling substance or staining reagent, however, in order to prevent the extracellular microparticles from being washed away, it is preferable that such washing not be performed.

Hereinafter, an example of an analysis sample preparation method will be shown, taking a case in which nucleic acids, proteins, lipids and sugar chains are stained in the population of extracellular microparticles as an example. However, it is needless to say that the analysis sample preparation method in the present embodiment is not limited to the following examples.

First, a sample containing a population of extracellular microparticles is optionally diluted or concentrated to an appropriate concentration. Then, a lipid staining reagent, for example, CellMask, is added, and the mixture is left, for example, at 0 °C or higher and 30 °C or lower for 1 minute or longer and 30 minutes or shorter. Next, the remaining nucleic acid staining reagent, protein staining reagent, and sugar chain staining reagent are added, and the mixture is left, for example, at 0 °C or higher and 30 °C or lower for 5 minutes or longer and 60 minutes or shorter. In this manner, when staining with a lipid staining reagent is performed in advance, the efficiency of lipid staining tends to be improved.

The sample containing a population of extracellular microparticles is not particularly limited, and examples thereof include a biological sample and biological cleaning liquids. Examples of non-limiting samples include peripheral blood mononuclear cells (PBMCs) and bronchoalveolar lavage fluids (BALF).

### (Light Emission)

In FIG. 1, next, the population of extracellular microparticles is irradiated with light. The light irradiation method is not particularly limited as long as scattered light and luminescent emissions can be detected for each particle from the population of extracellular microparticles during light detection described below. For example, the population of extracellular microparticles may be irradiated at once, or one particle or several particles (for example, about 1 to 10 particles) may be irradiated at a time while flowing extracellular microparticles through a flow path. Here, one particle may be irradiated with one type or one or more types of light beams. In addition, white light or monochromatic light may be irradiated. When monochromatic light is irradiated, the wavelength of the light to be irradiated may be selected according to the wavelength of excitation light of components of the extracellular microparticles or the labeling substances bound to the components. For example, when the population of extracellular microparticles is labeled with a plurality of types of labeling substances with different excitation light wavelengths, a plurality of monochromatic light beams corresponding to the excitation light wavelengths may be irradiated. The method of irradiating the population of extracellular microparticles for each particle with light is not particularly limited, and for example, a method using a flow cytometer may be used. The light to be irradiated may be a monochromatic light laser. The wavelength of the light to be irradiated is not particularly limited, and may be, for example, in a range of 400 to 600 nm.

### (Light Detection)

When the extracellular microparticles are irradiated with light as described above, in each extracellular microparticle, scattered light and luminescent emissions (typically fluorescence light) derived from components of the extracellular microparticle or labeling substances that bind to the components are generated. In the analysis method according to the present embodiment, the scattered light and the luminescent emissions are detected for each extracellular microparticle. In the analysis method according to the present embodiment, in the light detection, a sample is prepared such that a plurality of luminescent emissions having different peak wavelengths generated from the components of the extracellular microparticle or labeling substances that bind to the components are detected, or such a sample is prepared.

Examples of scattered light to be detected include forward scattered light and side scattered light. In addition, in addition to normal scattered light or instead of normal scattered light, some polarized light elements of the scattered light may be detected. To broaden the size range of extracellular microparticles that can be detected, for example, forward scattered light (FSC) and polarized forward scattered light (FSC-perp) may be detected simultaneously. At least two or more of side scattered light (SSC), forward scattered light (FSC) and polarized forward scattered light (FSC-perp), or all of them may be detected simultaneously.

The amount of luminescent emissions derived from the extracellular microparticles is detected in the number equal to the sum of the number of types of components that generate autoluminescence and the number of types of labeling substances contained in the extracellular microparticles. That is, according to the present embodiment, a plurality of luminescent emissions having different peak wavelengths are detected.

A suitable photodetector may be used to detect the scattered light and the luminescent emissions. One photodetector, or one or more photodetectors may be used. When one photodetector is used, a spectrum analyzer may be used to detect a plurality of luminescent emissions having different peak wavelengths. When one or more photodetectors are used, a suitable spectroscopic element may be used to spectrally separate the scattered light and the luminescent emissions generated by the above light emission according to respective peak wavelengths, and each separated light beam may be detected by a detector corresponding to each light beam.

Examples of photodetectors include a photomultiplier tube and a photodiode. The photodetector may be one that is installed in a flow cytometer.

### (Creation of Scatter Diagram)

As described above, by light irradiation and light detection, for each extracellular microparticle in a population of extracellular microparticles, the scattered light intensity and the emission light intensities of a plurality of luminescent emissions having different peak wavelengths are obtained. For example, when the population of extracellular microparticles is stained with three staining reagents having emission light wavelengths of λ₁, λ₂, and λ₃, information about, for example, the intensity I_{FSC} of forward scattered light, the intensity I_{p-FSC} of polarized forward scattered light, the intensity I_{SSC} of side scattered light, and the intensities I_{λ1}, I_{λ2}, and I_{λ3} of three luminescent emissions having wavelengths of λ₁, λ₂, and λ₃ are obtained for each extracellular microparticle. That is, for each extracellular microparticle, a vector whose components are the scattered light intensity and the emission light intensities is obtained. Explaining with the above example, for each extracellular microparticle, a vector (I_{FSC}, I_{p-FSC}, I_{SSC}, I_{λ1}, I_{λ2}, I_{λ3}) is obtained.

The analysis method according to the present embodiment includes, by an information processing device or an information processing unit of an analysis device (hereinafter simply referred to as an “information processing device”), generating a feature vector corresponding to the extracellular microparticle based on the scattered light intensity and the emission light intensities of a plurality of luminescent emissions having different peak wavelengths which are detected; and creating a first scatter diagram by mapping at least a portion of the extracellular microparticles contained in the population of extracellular microparticles in two or three dimensions based on the similarity of representative vectors composed of part or all of the elements of the feature vectors, and labeling each of the mapped extracellular microparticles based on at least a plurality of the elements corresponding to the luminescent emissions among the elements of the representative vectors. In addition, the mapped extracellular microparticles are classified into two or more clusters based on the first scatter diagram. Hereinafter, information processing in the analysis method of the present embodiment will be described in detail with reference to FIG. 2. In addition, the above example in which the population of extracellular microparticles is stained with three staining reagents having emission light wavelengths of λ₁, λ₂, and λ₃, and a vector (I_{FSC}, I_{p-FSC}, I_{SSC}, I_{λ1}, I_{λ2}, I_{λ3}) is obtained is appropriately used.

First, the information processing device generates feature vectors corresponding to the extracellular microparticles based on the detected scattered light intensity and the emission light intensities of a plurality of luminescent emissions having different peak wavelengths. The feature vector may be a vector whose elements include the detected scattered light intensity and the emission light intensities of the plurality of luminescent emissions having different peak wavelengths, or may be a vector obtained by applying appropriate processing to the vector. Examples of such processing include processing that does not substantially change the magnitude relationship between elements in a vector before processing such as binning processing of converting continuous values into discrete values or normalization processing. The processing that does not substantially change the magnitude relationship between elements in a vector before processing is processing that does not change the magnitude relationship between elements in a vector before processing or discretizes elements in a vector before processing. In the above example, the feature vector may be, for example, a vector (I_{FSC}, I_{p-FSC}, I_{SSC}, I_{λ1}, I_{λ2}, I_{λ3}), a vector (I_{FSC}, I_{p-FSC}, I_{SSC}, 1, 1, 0), or a vector (I_{FSC}, I_{λ1}, I_{λ2}, I_{λ3}). Here, the vector (I_{FSC}, I_{p-FSC}, I_{SSC}, 1, 1, 0) is an example of a vector that is processed to indicate, with 0 or 1, whether a sample is stained with each of the three staining reagents, depending on the values of the intensities I_{λ1}, I_{λ2}, and I_{λ3} of three luminescent emissions having wavelengths of λ₁, λ₂, and λ₃. In addition, the vector (I_{FSC}, I_{λ1}, I_{λ2}, I_{λ3}) is an example of a vector from which the intensity I_{FSC} of most characteristic scattered light is extracted according to the intensity I_{FSC} of forward scattered light, the intensity I_{p-FSC} of polarized forward scattered light, and the intensity of the intensity I_{SSC} of side scattered light. In this case, the extracted I_{FSC} element may correspond to the size of the extracellular microparticles. Hereinafter, an example in which the vector (I_{FSC}, I_{p-FSC}, I_{SSC}, I_{λ1}, I_{λ2}, I_{λ3}) is used as the feature vector will be used.

Next, the information processing device generates representative vectors composed of part or all of the elements of the feature vectors. The representative vector preferably includes at least an element corresponding to the scattered light intensity and an element corresponding to luminescent emissions among the elements of the feature vector. In the above example, the representative vector is, for example, a vector (I_{FSC}, I_{p-FSC}, I_{λ1}, I_{λ2}, I_{λ3}).

Which of the elements of the feature vector is used to generate the representative vector may be set by a user of the information processing device or may be set by the information processing device based on a predetermined algorithm. The information processing device generates a representative vector having set components of the feature vector as components for each extracellular microparticle. The representative vector may be the same vector as the feature vector.

Next, the information processing device determines extracellular microparticles to be mapped from among the population of extracellular microparticles. In this case, all extracellular microparticles in the population of extracellular microparticles may be mapped, or some of the extracellular microparticles contained in the population of extracellular microparticles may be mapped. The extracellular microparticles to be mapped are determined based on, for example, feature vectors or representative vectors. The information processing device may map all extracellular microparticles in the population of extracellular microparticles without determining the extracellular microparticles to be mapped.

The information processing device may exclude from mapping, for example, extracellular microparticles in which all of the elements corresponding to the luminescent emissions among the elements of the representative vectors are equal to or less than a threshold. The above example corresponds to excluding extracellular microparticles in which all of I_{λ1}, I_{λ2}, and I_{λ3} are equal to or less than a threshold from mapping. This aspect corresponds to excluding extracellular microparticles that are not labeled or stained or that have a low degree of labeling or staining within the population of extracellular microparticles from mapping. In this manner, when extracellular microparticles that are not labeled or stained or that have a low degree of labeling or staining are excluded from mapping, cluster analysis to be described below can be performed with higher accuracy.

Alternatively, the information processing device may map only extracellular microparticles in which predetermined elements among the elements corresponding to the luminescent emissions among the elements of the representative vectors are equal to or larger than a threshold or equal to or less than a threshold or may exclude from mapping the extracellular microparticles. This aspect corresponds to adding to mapping or excluding from mapping extracellular microparticles having a high or low content of a predetermined component within the population of extracellular microparticles.

The above threshold may be set by a user of the information processing device or may be set by the information processing device based on a predetermined algorithm. For example, an unlabeled or unstained population of extracellular microparticles and a labeled or stained population of extracellular microparticles are prepared, from the same population of extracellular microparticles, and then feature vectors or representative vectors may be generated for the unlabeled or unstained population of extracellular microparticles, and the above threshold may be set based on an element corresponding to the emission light intensity in the feature vectors or representative vectors. In this case, the threshold may be the maximum value, the average value, the mode value, the value of the average value+2σ, the value of the average value+3σ, or the value of the average value+4σ of the elements corresponding to the intensities of luminescent emissions in feature vectors or representative vectors in the unlabeled or unstained population of extracellular microparticles.

Next, the information processing device creates a two-dimensional or three-dimensional scatter diagram by mapping, in two or three dimensions, the extracellular microparticles that are mapping targets in the above, based on the similarity of respective representative vectors. For example, the mapping may be performed by reducing the dimension of the representative vector corresponding to each extracellular microparticle or may be performed by machine learning. More specifically, techniques such as T-distributed Stochastic Neighbor Embedding (t-SNE), SNE, Uniform Manifold Approximation and Projection (UMAP), and principal component analysis (PCA) can be used. In the created two-dimensional or three-dimensional scatter diagram, a plurality of extracellular microparticles are plotted based on the similarity of the representative vectors. That is, extracellular microparticles with similar representative vectors (corresponding to similar sizes and degrees of staining) are plotted adjacent to each other, and extracellular microparticles with different representative vectors (corresponding to different sizes and degrees of staining) are plotted apart from each other.

Next, the information processing device labels respective points corresponding to extracellular microparticles in the scatter diagram based on at least a plurality of the elements corresponding to the luminescent emissions among the elements of the representative vectors. Examples of labels to be assigned include the type of the component or labeling substance from which luminescent emission is detected, and the peak wavelength of luminescent emission. For example, labeling may be performed with a component, labeling substance, or staining reagent corresponding to the component with the strongest intensity among the elements corresponding to the intensities of luminescent emissions in the feature vectors or representative vectors, and the following method may be used for labeling. That is, a method may be used in which it is determined, for each labeling substance, whether each extracellular microparticle in the population of extracellular microparticles is labeled with the labeling substance, a target substance with the smallest number of labeled extracellular microparticles is set as a first labeling substance, and a target substance with the second smallest number of labeled extracellular microparticles is set as a second labeling substance, and then the extracellular microparticles stained with the first labeling substance are labeled with the first labeling substance, the extracellular microparticles stained with the second labeling substance are labeled with the second labeling substance, and the labeling is repeated the number of times equal to the number of labeling substances. In this labeling, the labels to be assigned to the extracellular microparticles may be set by a user of the information processing device or may be set by the information processing device based on a predetermined algorithm.

As described above, at least some of the extracellular microparticles contained in the population of extracellular microparticles are mapped and a scatter diagram labeled with a predetermined label can be created. Hereinafter, this scatter diagram will be referred to as a first scatter diagram.

Here, although an example of information processing in the analysis method for extracellular microparticles according to the present embodiment will be described with reference to FIG. 2, it is needless to say that the information processing in the analysis method of the present embodiment is not limited to the above example. For example, before or during the above information processing, data processing such as feature engineering for machine learning may be performed. For example, binning processing may be performed on each feature vector obtained from a population of extracellular microparticles based on the scattered light intensity and/or the emission light intensity derived from each labeling substance.

### (Clustering)

The analysis method of the present embodiment will be described again with reference to FIG. 1. Next, as described above, based on the first scatter diagram created by the information processing device, the plurality of extracellular microparticles mapped on the first scatter diagram are classified into two or more clusters. The classification into two or more clusters (hereinafter referred to as clustering) may be performed based on the labels of the first scatter diagram. For example, clustering may be performed so that extracellular microparticles contained in a part where the same labels are concentrated in the first scatter diagram are included in the same cluster. Clustering may be performed by a user or may be performed by the information processing device based on a predetermined algorithm. Examples of algorithms used include k-means, k-means++, support vector machine (SVM), and kernel SVM.

The number of clusters to be generated is not particularly limited as long as it is 2 or more, and may be, for example, within the range of the number of detected luminescent emissions±5, the range of the number of detected luminescent emissions±4, the range of the number of detected luminescent emissions±3, the range of the number of detected luminescent emissions±2, the range of the number of detected luminescent emissions±1, or the same number as the number of detected luminescent emissions. Alternatively, when the number of elements corresponding to luminescent emissions among the elements of the representative vectors is n, clusters in the range of n±5, n±4, n±3, n±2, n±1, or n may be generated. Alternatively, when the number of labels assigned in the first scatter diagram is m, clusters in the range of m±5, m±4, m±3, m±2, m±1, or m may be generated.

The information processing device may record cluster information regarding clusters into which the extracellular microparticles belonging to the generated clusters are classified, in association with the feature vector corresponding to the extracellular microparticles.

As described above, according to the analysis method of the present embodiment, the population of extracellular microparticles with unknown characteristics can be analyzed and classified into two or more clusters. According to the analysis method of the present embodiment, for example, it is possible to clarify whether the number of extracellular microparticles having a predetermined characteristic increases or decreases depending on the presence of diseases, and it is possible to identify extracellular microparticles that serve as biomarkers for disease onset, disease cause, and/or prognosis prediction. In addition, the effectiveness of vaccines and therapeutic agents can be verified by confirming that the vaccines and therapeutic agents reduce the number of extracellular microparticles which increases when a patient has a disease.

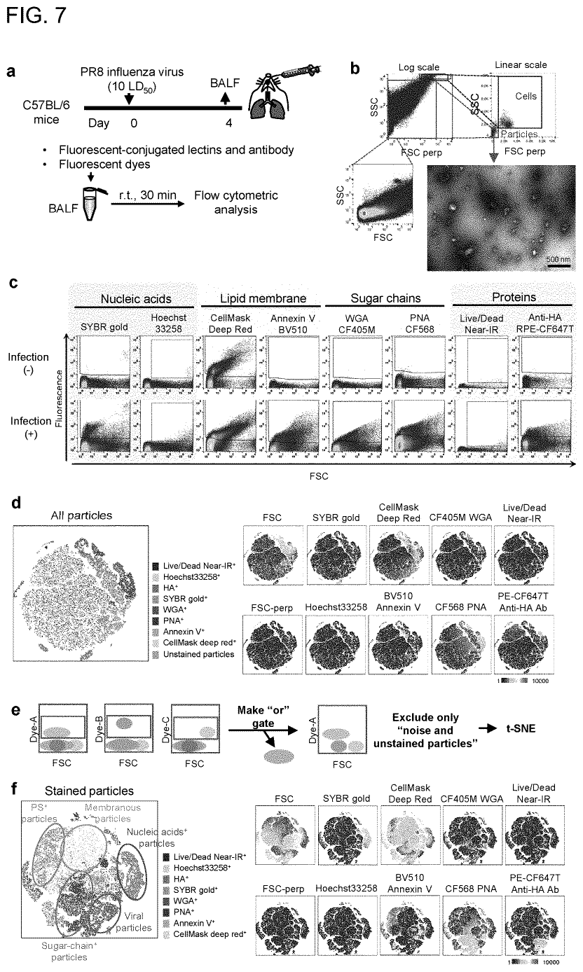

On the left side of FIG. 7f, an example of the results obtained by analyzing the population of extracellular microparticles by the analysis method of the present embodiment is shown. The diagram shown on the left side of FIG. 7f shows the results obtained when a population of extracellular microparticles is stained with 8 types of staining reagents, the population of extracellular microparticles is mapped based on scattered light and fluorescence derived from the staining reagents and labeled with any one of the 8 types of staining reagents, and the population of extracellular microparticles is classified into at least 5 clusters. In this manner, according to the analysis method of the present embodiment, the population of extracellular microparticles can be comprehensively analyzed, the population of extracellular microparticles can be classified into two or more clusters, and the overall composition of the population of extracellular microparticles can be clarified.

### (Identification of Cluster)

The analysis method of the present embodiment may further include identifying characteristics of at least one cluster of the clusters obtained by the clustering. The identification may include creating a second scatter diagram by the information processing device and identifying characteristics of a cluster based on the first and second scatter diagrams. Hereinafter, creation of the second scatter diagram and identification of characteristics of a cluster based on the first and second scatter diagrams will be described in detail.

The second scatter diagram is a scatter diagram in which at least a portion of the plurality of extracellular microparticles contained in the population of extracellular microparticles are mapped in the same manner as the first scatter diagram, and one of a plurality of the elements corresponding to the luminescent emissions among the elements of the representative vectors is visualized in each of the mapped extracellular microparticles. That is, the second scatter diagram differs from the first scatter diagram in that, instead of labeling based on at least a plurality of the elements corresponding to the luminescent emissions among the elements of the representative vectors, information on the magnitude value of one of a plurality of the elements corresponding to the luminescent emissions among the elements of the representative vectors, that is, the intensity of one of a plurality of the luminescent emissions, is visualized and assigned.

In the second scatter diagram, the visualized information may be visualized by a change in color or may be visualized by the size of points corresponding to extracellular microparticles. For example, the second scatter diagram is a heat map showing the degree of staining of the extracellular microparticles with a predetermined staining reagent.

The second scatter diagram is created by the information processing device. The information processing device creates a scatter diagram by mapping at least some of the extracellular microparticles contained in the population of extracellular microparticles in two or three dimensions in the same manner as the first scatter diagram. Next, the information processing device assigns information on the magnitude value of one of a plurality of the elements corresponding to the luminescent emissions among the elements of the representative vectors to respective points corresponding to extracellular microparticles in the scatter diagram. In the above example in which the vector (I_{FSC}, I_{p-FSC}, I_{λ1}, I_{λ2}, I_{λ3}) is obtained as the representative vector, the information processing device assigns information on the magnitude of any of I_{λ1}, I_{λ2}, and I_{λ3} to respective points corresponding to extracellular microparticles in the scatter diagram.

The information processing device may create a plurality of second scatter diagrams in which the types of luminescent emissions whose intensities are visualized are different from each other. That is, when the representative vector includes an element corresponding to first luminescent emission and an element corresponding to second luminescent emission, the information processing device may create a second scatter diagram “A” in which the element corresponding to the first luminescent emission is visualized and a second scatter diagram “B” in which the element corresponding to the second luminescent emission is visualized. In the above example in which the vector (I_{FSC}, I_{p-FSC}, I_{λ1}, I_{λ2}, I_{λ3}) is obtained as the representative vector, the information processing device may create three types of second scatter diagrams in which information on the magnitude of I_{λ1}, I_{λ2}, and I_{λ3} is assigned to respective points in the scatter diagram.

Based on the first and second scatter diagrams, the characteristics of the clusters may be identified by comparing the first and second scatter diagrams. The characteristics of the clusters may be identified by comparing the first scatter diagram with a plurality of second scatter diagrams. For example, using a case in which the vector (I_{FSC}, I_{p-FSC}, I_{λ1}, I_{λ2}, I_{λ3}) is obtained as the representative vector, and three types of second scatter diagrams in which information on the magnitude of I_{λ1}, I_{λ2}, and I_{λ3} is assigned to respective points in the scatter diagram are created as an example, with reference to the three types of second scatter diagrams, it is possible to confirm the degree of staining by three staining reagents having emission light wavelengths of λ₁, λ₂, and λ₃ in at least one cluster in the first scatter diagram, and it is possible to identify characteristics such as the content of each component of the extracellular microparticles.

On the right side of FIG. 7f, an example when a plurality of second scatter diagrams are created from the population of extracellular microparticles by the analysis method of the present embodiment is shown. The diagram shown on the right side of FIG. 7f shows a total of 10 heat maps in which the population of extracellular microparticles is stained with 8 types of staining reagents, the population of extracellular microparticles is mapped based on scattered light and fluorescence derived from the staining reagents, and the scattered light intensity (2 types) or the fluorescence intensity (8 types) is visualized. By comparing these heat maps with the first scatter diagram shown on the left side of FIG. 7f, it is possible to easily confirm which components are included in large amounts in the clusters identified in the first scatter diagram.

### (Analysis Using Analysis Data)

According to the above analysis method, it is possible to acquire, for a plurality of extracellular microparticles, a feature vector showing characteristics of the extracellular microparticles in association with cluster information regarding clusters into which the extracellular microparticles are classified. Therefore, when a population of extracellular microparticles is analyzed by the above analysis method and analysis data including a combination of a plurality of feature vectors and cluster information is acquired, additional analysis can be performed using the analysis data as training data. Therefore, the present embodiment can provide training data including a combination of a plurality of feature vectors and cluster information obtained by the above analysis method and a trained model trained using the training data.

For example, an analysis method for extracellular microparticles according to another aspect of the present embodiment includes irradiating an extracellular microparticle with incident light, detecting light scattered from the incident light and a plurality of luminescent emissions having different peak wavelengths generated from components of the extracellular microparticles or labeling substances bound to the components in response to the incident light, and thus acquiring a feature vector corresponding to the extracellular microparticle; and analyzing characteristics of the extracellular microparticle based on analysis data obtained by analyzing a population of extracellular microparticles by the analysis method described above in detail and the acquired feature vector.

This analysis method is a method in which a feature vector is acquired for an extracellular microparticle in the same manner as in the analysis method shown in FIG. 1, and characteristics of the extracellular microparticle from which the feature vector is acquired are analyzed based on the feature vector and existing analysis data including a combination of a plurality of feature vectors and cluster information. This analysis method makes it possible to clarify, for example, a cluster into which the extracellular microparticle having a known feature vector is classified.

This analysis method may include generating a model (extracellular microparticle characteristic prediction model) by machine learning using analysis data obtained by analyzing the population of extracellular microparticles by the analysis method described above in detail as training data. For model generation, techniques such as convolutional neural networks (CNNs), decision trees, random forests, naive bayes methods, and support vector machines may be used.

### Modified Example

An analysis method for extracellular microparticles according to another aspect of the present embodiment includes detecting light scattered from incident light and a plurality of luminescent emissions having different peak wavelengths generated from components of the extracellular microparticle or labeling substances bound to the components in response to the incident light, and thus generating feature vectors corresponding to the respective extracellular microparticles; and classifying the population of extracellular microparticles into two or more clusters based on the similarity of representative vectors composed of some or all of the elements of the feature vectors.

This analysis method differs from the analysis method described above in detail in that the population of extracellular microparticles is classified into two or more clusters based on the similarity of representative vectors without creating a first scatter diagram. As the clustering method, hierarchical clustering and non-hierarchical clustering can be used, and for example, a single-link method, a Ward method, a k-means method, and a k-means++ method may be used. The other points are the same as those of the analysis method described above in detail, and description thereof will be omitted.

### [Separation Method]

In addition, when analysis data including a combination of a plurality of feature vectors and cluster information is acquired by the analysis method of the present embodiment, it is possible to separate extracellular microparticles having a specific characteristic from the population of extracellular microparticles. In such separation, specific extracellular microparticles may be separated from the population of extracellular microparticles analyzed by the analysis method of the present embodiment, and specific extracellular microparticles may be separated from a population of extracellular microparticles different from the population of extracellular microparticles analyzed by the analysis method of the present embodiment.

For example, a separation method for extracellular microparticles according to another aspect of the present embodiment includes, for an extracellular microparticle in a population of extracellular microparticles, detecting light scattered from incident light and a plurality of luminescent emissions having different peak wavelengths generated from components of the extracellular microparticle or labeling substances bound to the components in response to the incident light, and thus acquiring a feature vector corresponding to the extracellular microparticle; determining whether the extracellular microparticle has a predetermined characteristic based on analysis data obtained by analyzing a population of extracellular microparticles by any of the analysis methods according to the present embodiment and the acquired feature vector; and separating, when it is determined that the extracellular microparticle has the predetermined characteristic, the extracellular microparticle from the population of extracellular microparticles.

Such a separation method is a method in which a feature vector is acquired for extracellular microparticles in the same manner as in the analysis method shown in FIG. 1, it is determined whether extracellular microparticles from which the feature vector is acquired have a predetermined characteristic based on the feature vector and analysis data including a combination of a plurality of feature vectors and cluster information, and extracellular microparticles determined to have the predetermined characteristic are separated from the population of extracellular microparticles.

In this separation method, the population of extracellular microparticles from which predetermined extracellular microparticles are separated may be the same as or different from the population of extracellular microparticles used to acquire analysis data. For example, a first population of extracellular microparticles is analyzed by the analysis method of the present embodiment to acquire analysis data including a combination of a plurality of feature vectors and cluster information, and predetermined extracellular microparticles may be separated from a second population of extracellular microparticles based on the analysis data, and when the first population of extracellular microparticles is analyzed by the analysis method of the present embodiment to acquire analysis data including a combination of a plurality of feature vectors and cluster information, and predetermined extracellular microparticles may be separated based on the analysis data, from the first population of extracellular microparticles.

In this separation method, a user may set characteristics of extracellular microparticles to be separated. For example, the user may instruct the information processing device to separate extracellular microparticles that belong to one of the clusters of extracellular microparticles determined by the analysis method of the present embodiment. In this case, the information processing device performs analysis based on the analysis data regarding what characteristics the extracellular microparticles belonging to the designated cluster have. For example, the information processing device refers to the feature vectors or representative vectors of extracellular microparticles belonging to the designated cluster, and sets the conditions under which extracellular microparticles should be separated based on the range of the elements of the feature vectors or representative vectors. Alternatively, the information processing device may generate a prediction model for predicting whether a given extracellular microparticle belongs to the designated cluster. When the prediction model is generated, data including the feature vectors or representative vectors of extracellular microparticles belonging to the designated cluster and cluster information may be used as training data. The prediction model may be a regression model or a model generated by machine learning. For model generation, techniques such as convolutional neural networks (CNNs), decision trees, random forests, naive bayes methods, and support vector machines may be used.

More specifically, the extracellular microparticles to be separated may be determined as follows. First, the user instructs the information processing device to separate extracellular microparticles that belong to one of the clusters of extracellular microparticles determined by the analysis method of the present embodiment. The information processing device identifies the components of the labeled or stained extracellular microparticles for the extracellular microparticles of the designated cluster based on analysis data including a combination of a plurality of feature vectors and cluster information obtained by analyzing the population of extracellular microparticles by the analysis method of the present embodiment. The presence of labeling or staining may be determined based on the emission light intensity derived from the labeling substance or staining reagent or the element of corresponding feature vectors or representative vectors, and for example, it may be determined that extracellular microparticles in which the emission light intensity or the element of corresponding feature vectors or representative vectors is equal to or larger than a threshold are labeled or stained. Next, the information processing device acquires a feature vector related to the extracellular microparticle in the population of extracellular microparticles used for separation, and identifies the labeled or stained components in the extracellular microparticle based on the feature vectors. When the labeled or stained components of the extracellular microparticle are the same as the labeled or stained components of the extracellular microparticles of the designated cluster, the information processing device determines that the extracellular microparticles are separation targets.

The method of separating extracellular microparticle determined to have a predetermined characteristic from a population of extracellular microparticles is not particularly limited as long as it is a method with which an extracellular microparticle can be separated at the single-particle level, and examples thereof include a method using a flow cytometer. For example, a method in which a droplet containing an extracellular microparticle are formed, the droplet containing the extracellular microparticle to be separated is charged, and only the droplet containing the extracellular microparticle to be separated is electrically separated may be used.

In this manner, a plurality of extracellular microparticles having predetermined characteristics can be separated to acquire a new population of extracellular microparticles. The obtained population of extracellular microparticles may be analyzed again by the analysis method of the present embodiment to perform additional cluster analysis, or may be subjected to other analyses such as PCR.

### [Analysis Device]

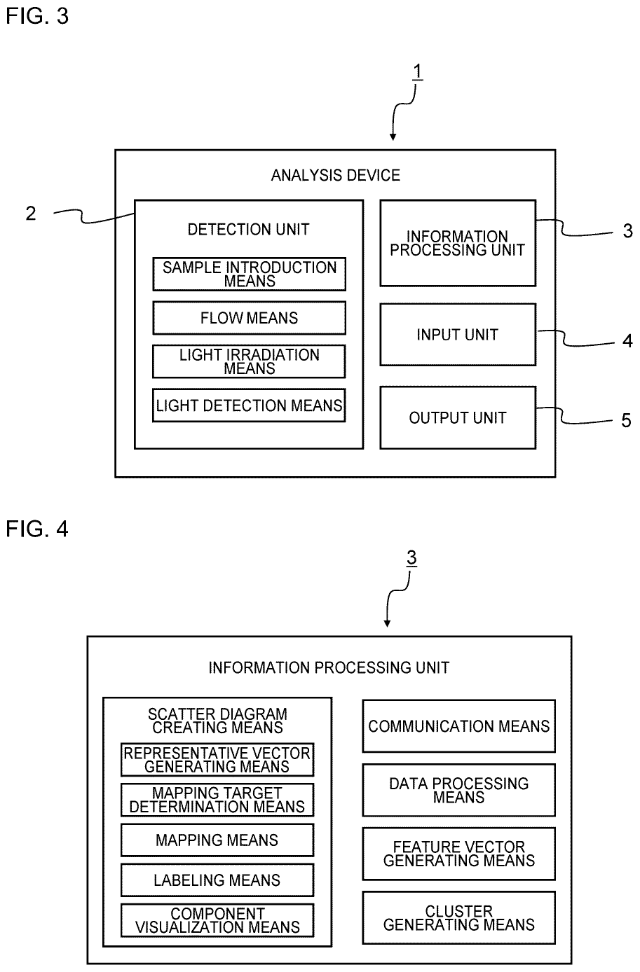

FIG. 3 is a diagram showing an example of a configuration of an analysis device for extracellular microparticles according to the present embodiment. As shown in FIG. 3, an analysis device 1 according to the present embodiment includes a detection unit 2 configured to detect, for each extracellular microparticle in a population of extracellular microparticles, light scattered from incident light and a plurality of luminescent emissions having different peak wavelengths generated from components of the extracellular microparticle or labeling substances bound to the components in response to the incident light; and an information processing unit 3 including a feature vector generating means for generating a feature vector corresponding to the extracellular microparticle in the population of extracellular microparticles based on the detected scattered light and the plurality of luminescent emissions, a scatter diagram creating means for creating a first scatter diagram by mapping at least a portion of the extracellular microparticles contained in the population of extracellular microparticles in two or three dimensions based on the similarity of representative vectors composed of some or all of the elements of the acquired feature vector, and labeling each of the mapped extracellular microparticles based on at least a plurality of the elements corresponding to the detected luminescent emissions among the elements of the representative vectors, and a cluster generating means for generating two or more clusters to which the extracellular microparticles belong by assigning cluster information to each of the extracellular microparticles mapped on the first scatter diagram. In addition, the analysis device 1 according to the present embodiment includes an input unit 4 configured to allow a user to input an instruction to the information processing unit and the detection unit and input data, and an output unit 5 configured to output the analysis result.

Hereinafter, respective configurations will be described.

The detection unit 2 detects, for each extracellular microparticle in a population of extracellular microparticles, light scattered from incident light and a plurality of luminescent emissions having different peak wavelengths generated from components of the extracellular microparticle or labeling substances bound to the components in response to the incident light. In FIG. 3, the detection unit 2 includes a sample introduction means for introducing a sample to be analyzed, a flow means for causing the introduced sample to flow through a flow path and creating a state in which extracellular microparticles flow continuously and substantially in a line, a light irradiation means for irradiating the extracellular microparticles that flow continuously and substantially in a line by the flow means, and a light detection means for detecting scattered light and a plurality of luminescent emissions having different peak wavelengths from the extracellular microparticles to which light is irradiated. The detection unit 2 may have any configuration as long as it can detect scattered light and a plurality of luminescent emissions having different peak wavelengths for each extracellular microparticle in a population of extracellular microparticles as described above, and for example, a commercially available flow cytometer may be used as the detection unit.

The flow means causes the introduced sample to flow through a flow path and creates a state in which extracellular microparticles flow continuously and substantially in a line. The flow means may include a flow cell having a sample inlet and a sample outlet, and a pressure applying means such as a pump for introducing a sample into the flow cell. The pressure applied by the pressure applying means is adjusted within a range in which a laminar flow occurs in the flow cell.

The light irradiation means irradiates with light having an excitation wavelength of components contained in extracellular microparticles or labeling substances that bind to the components. The light to be irradiated may be white light or monochromatic light. When the population of extracellular microparticles is labeled with a plurality of types of labeling substances with different excitation light wavelengths, the detection unit 2 may include a plurality of light irradiation means for irradiating a plurality of types of monochromatic light beams corresponding to the wavelength of the excitation light.

The light detection means detects scattered light and a plurality of luminescent emissions having different peak wavelengths generated from extracellular microparticles for each particle. The light detection means may include, for example, spectrometers such as a diffraction grating, a bandpass filter, and a notch filter, light collecting elements such as a lens, detectors such as a photomultiplier tube and a photodiode, and polarizing elements for detecting polarized light elements. More specifically, the light detection means may include a light collecting element; a spectrometer and a detector for detecting scattered light; a spectrometer, a polarizing element and a detector for detecting polarized scattered light; and a plurality of spectrometers and detectors for detecting a plurality of luminescent emissions having different peak wavelengths.

The input unit 4 receives an input to the analysis device 1. The input unit 4 includes, for example, a keyboard, a mouse, a microphone, and/or a touch panel for receiving an input from a user. The analysis device 1 causes the detection unit 2, the information processing unit 3, and the output unit 5 to execute respective functions based on information input from the input unit 4. In addition, the input unit 4 may receive analysis data obtained by analyzing a population of extracellular microparticles by the analysis method described above in detail.

The output unit 5 displays the analysis results of the analysis device 1. The output unit 5 includes, for example, a liquid crystal display or an organic EL display.

FIG. 4 is a block diagram showing an example of a functional configuration of the information processing unit 3. In FIG. 4, the information processing unit 3 includes a communication means for communicating with the detection unit 2, the input unit 4 and the output unit 5, a data processing means for processing data obtained from the detection unit 2, a feature vector generating means for generating a feature vector from the processed data, a scatter diagram creating means for creating a scatter diagram based on the feature vector, and a cluster generating means for generating two or more clusters to which the plurality of extracellular microparticles belong by assigning cluster information to each of the extracellular microparticles mapped on the scatter diagram.

The information processing unit 3 may be integrated with the detection unit 2, the input unit 4, and/or the output unit 5 to constitute the analysis device 1, or may be connected to the detection unit 2, the input unit 4, and/or the output unit 5 via a wireless or wired communication to constitute the analysis device 1. The information processing unit 3 communicates with other components of the analysis device 1 via the communication means. For example, the information processing unit 3 may receive detection data from the detection unit through communication from the input unit, transmit the analysis results to the output unit, and display them on the output unit. The communication means is not particularly limited, and may be processing within the device or wireless communication or wired communication.

The data processing means processes the detection data received from the detection unit 2 for subsequent analysis. For example, the extracellular microparticles may be associated with the detected scattered light intensity and emission light intensity based on the detection data. The data processing means may perform light emission crosstalk correction. The light emission crosstalk correction is correction for reducing the influence of a plurality of luminescent emissions having different peak wavelengths on each other's detection intensities. For example, when first luminescent emission exhibits broad light emission and its peak wavelength overlaps that of second luminescent emission having a different peak wavelength, the correction corresponds to subtracting the intensity caused by the first luminescent emission from the second luminescent emission intensity. In addition, the data processing means may perform binning processing on each feature vector generated by the feature vector generating means based on the scattered light intensity and/or the emission light intensity derived from each labeling substance.

The feature vector generating means generates a feature vector indicating the characteristics of the extracellular microparticle based on the detection data received from the detection unit or the data processed by the data processing means. The feature vector may be a vector whose elements include the detected scattered light intensity and the emission light intensities of a plurality of luminescent emissions having different peak wavelengths or may be a vector obtained by applying appropriate processing to the vector. Examples of such processing include processing that does not substantially change the magnitude relationship between elements in a vector before processing such as binning processing of converting continuous values into discrete values or normalization processing.

The scatter diagram creating means creates a first scatter diagram by mapping at least a portion of the extracellular microparticles contained in the population of extracellular microparticles in two or three dimensions based on the similarity of representative vectors composed of some or all of the elements of the feature vectors, and labeling each of the mapped extracellular microparticles based on at least a plurality of the elements corresponding to the luminescent emissions among the elements of the representative vectors. In addition, the scatter diagram creating means may create a second scatter diagram by mapping at least a portion of the extracellular microparticles contained in the population of extracellular microparticles in the same manner as the first scatter diagram, and visualizing one of a plurality of the elements corresponding to the luminescent emissions among the elements of the representative vectors in each of the mapped extracellular microparticles.

In FIG. 4, the scatter diagram creating means includes a representative vector generating means for generating a representative vector composed of some or all of the elements of the feature vectors; a mapping target determination means for determining extracellular microparticles to be mapped from the population of extracellular microparticles; a mapping means for mapping a plurality of extracellular microparticles determined to be mapping targets based on the similarity of the representative vectors in two or three dimensions; a labeling means for labeling each of the mapped extracellular microparticles; and a component visualization means for visualizing one of a plurality of the elements corresponding to the luminescent emissions among the elements of the representative vectors in each of the mapped extracellular microparticles.

The representative vector generating means generates a representative vector composed of some or all of the elements of the feature vector, for example, in response to an instruction from the input unit or based on a predetermined algorithm. For example, the representative vector generating means may generate a representative vector by deleting elements, whose values are equal to or less than a threshold, or equal to or larger than a threshold for all extracellular microparticles, from the feature vector.

The mapping target determination means determines, for example, extracellular microparticles to be mapped based on the feature vectors or representative vectors. For example, extracellular microparticles in which all of the elements corresponding to the luminescent emissions among the elements of the representative vectors are equal to or less than a threshold may be excluded from mapping, or only extracellular microparticles in which predetermined elements among the elements corresponding to the luminescent emissions among the elements of the representative vectors are equal to or larger than a threshold or equal to or less than a threshold may be mapped and the extracellular microparticles may be excluded from mapping.

The mapping means performs mapping in two or three dimensions based on the similarity of the representative vectors. For example, the mapping may be performed by reducing the dimension of the representative vector corresponding to each extracellular microparticle or may be performed by machine learning. The specific technique is as described in detail in the analysis method of the present embodiment.

The labeling means labels respective points corresponding to the extracellular microparticles mapped by the mapping means based on at least a plurality of the elements corresponding to the luminescent emissions among the elements of the representative vectors. The labels to be assigned to the extracellular microparticles may be set in response to an instruction from the input unit or based on a predetermined algorithm. The specific technique is as described in detail in the analysis method of the present embodiment.

The component visualization means assigns information on the magnitude value of one of a plurality of the elements corresponding to the luminescent emissions among the elements of the representative vectors to respective points corresponding to the extracellular microparticles mapped by the mapping means. The component visualization means may visualize the information by color or may visualize the information by the size of each point corresponding to the extracellular microparticle. The components of the representative vectors visualized by the component visualization means may be set in response to an instruction from the input unit.

The cluster generating means generates two or more clusters to which the extracellular microparticles belong by assigning cluster information to each of the extracellular microparticles mapped on the first scatter diagram. The cluster generating means may assign the same cluster information to extracellular microparticles included in a predetermined range in the first scatter diagram. The range to which the same cluster information is assigned may be set in response to an instruction from the input unit or based on a predetermined algorithm. Examples of algorithms used include k-means, k-means++, support vector machine (SVM), and kernel SVM.

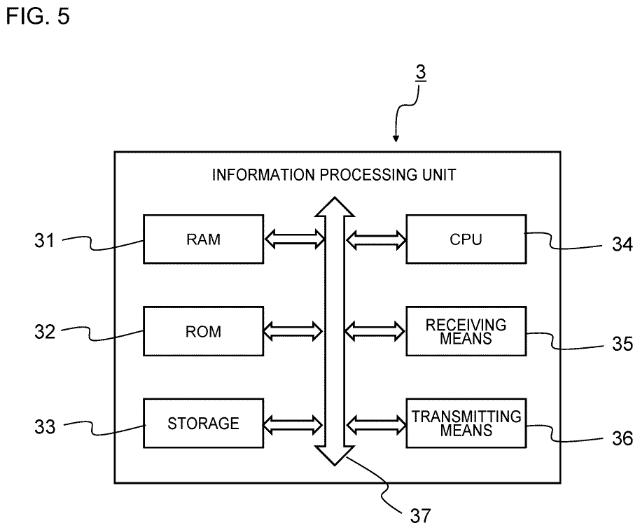

FIG. 5 is a block diagram showing an example of a physical configuration of the information processing unit 3. In FIG. 5, the information processing unit 3 includes a random access memory (RAM) 31, a read only memory (ROM) 32, a storage 33, a central processing unit (CPU) 34, a receiving means 35, a transmitting means 36, and a system bus 37 connecting these.

The RAM 31 is a data rewritable memory, and serves as a main memory. The RAM 31 may be composed of, for example, a semiconductor memory element, and stores programs such as applications executed by the CPU 34 and various types of data.

The ROM 32 is a data readable memory, and may be composed of, for example, a semiconductor memory element. The ROM 32 stores, for example, programs such as firmware, and data.

The storage 33 is a data rewritable memory, and serves as an auxiliary memory. The storage 33 may be composed of, for example, a semiconductor memory element, an optical disk, a hard disk drive (HDD), or a magnetic tape, and stores programs and various types of data.

The CPU 34 is a control unit that controls execution of programs stored in the RAM 31 and/or the ROM 32 and performs data calculation and processing. The information processing unit 3 realizes a function of analyzing extracellular microparticles under control of the CPU 34. The CPU 34 executes analysis based on the information and instructions received from the receiving means 35, transmits the analysis results to the transmitting means 36, and stores them in various storage devices such as the RAM 31 and the storage 33.

The receiving means 35 is a means for receiving the information and instructions from the detection unit 2 and the input unit 4 in the information processing unit 3. The transmitting means 36 is a means for transmitting the information and analysis results from the information processing unit 3 to the detection unit 2 and the output unit 5. The receiving means 35 and the transmitting means 36 may be wireless communication or wired communication.

In the information processing unit 3, the CPU 34 executes an analysis program for extracellular microparticles and thus realizes various functions described with reference to FIG. 4. Here, these physical configurations are only examples, and do not necessarily have to be independent configurations. For example, the information processing unit 3 may include a large-scale integration (LSI) in which the CPU 34, the RAM 31, the ROM 32 and/or the storage 33 are integrated.

### Modified Examples

While the analysis device for extracellular microparticles according to the present embodiment has been described above with reference to FIGS. 3 to 5, the analysis device for extracellular microparticles according to the present embodiment is not limited thereto, and can be variously modified without departing from the spirit and scope thereof. For example, the analysis device 1 may include a functional configuration or physical configuration that is not shown in FIGS. 3 to 5, or the functional configuration or physical configuration that is shown in FIGS. 3 to 5 may be omitted.

For example, the information processing unit 3 may include a model generating means for generating a model (extracellular microparticle characteristic prediction model) by machine learning using analysis data obtained by analyzing the population of extracellular microparticles by the analysis method described above in detail as training data or may include a classifying means for classifying a population of extracellular microparticles into two or more clusters based on the similarity of representative vectors without creating a first scatter diagram.

### [Separation Device]

A separation device for extracellular microparticles according to another aspect of the present embodiment includes a detection unit configured to irradiate an extracellular microparticle with incident light and detect light scattered from the incident light and a plurality of luminescent emissions having different peak wavelengths generated from components of the extracellular microparticle or labeling substances bound to the components in response to the incident light; an information processing unit including a feature vector generating means for generating a feature vector corresponding to the extracellular microparticle based on the detected scattered light and plurality of luminescent emissions and a determination means for determining whether the extracellular microparticle has a predetermined characteristic based on analysis data obtained by analyzing a population of extracellular microparticles by the analysis method according to any one of the above aspects and the acquired feature vector; and a separation unit configured to separate, when it is determined that the extracellular microparticle has a predetermined characteristic, the extracellular microparticles from the population of extracellular microparticles.

The separation device of the present embodiment further includes, for example, the separation unit configured to separate an extracellular microparticle from the population of extracellular microparticles, in addition to the configuration of the analysis device according to the present embodiment described above. In addition, the information processing unit further includes a determination means for determining whether an extracellular microparticle having a predetermined feature vector has a predetermined characteristic.

The determination means determines characteristics of extracellular microparticles to be separated in response to an instruction from the input unit or based on a predetermined algorithm, and determines whether extracellular microparticles having a predetermined feature vector have the same characteristics as the determined extracellular microparticles. For example, the determination means may set, based on analysis data obtained by analyzing a population of extracellular microparticles by the analysis method described above in detail, the conditions under which extracellular microparticles should be separated based on the range of the elements of the feature vectors or representative vectors, and may generate a prediction model for determining whether extracellular microparticles having a predetermined feature vector have a predetermined characteristic.

The separation unit separates the extracellular microparticles determined as separation targets by the determination means. The separation unit may include, for example, a droplet forming means for forming a droplet containing an extracellular microparticle, a charging means for charging the formed droplet, a deflecting plate that applies an electrostatic force to the charged droplet to change the trajectory of the droplet, and a collecting means for collecting a droplet containing an extracellular microparticle to be separated. Alternatively, the separation unit may include an opening and closing means for separating the extracellular microparticle to be separated by opening and closing a valve at the timing when the extracellular microparticle to be separated are detected.

### Appendix

The present invention includes the following embodiments.

[1]

An analysis method for extracellular microparticles, including:
for each extracellular microparticle in a population of extracellular microparticles, generating a feature vector corresponding to the extracellular microparticle by detecting light scattered from incident light and a plurality of luminescent emissions having different peak wavelengths, the luminescent emissions being generated from components of the extracellular microparticle or labeling substances bound to the components in response to the incident light;
creating a first scatter diagram by mapping, in two or three dimensions, at least a portion of the extracellular microparticles contained in the population of extracellular microparticles on the basis of the similarity of representative vectors composed of part or all of the elements of the feature vectors, and labeling each of the mapped extracellular microparticles on the basis of at least a plurality of the elements corresponding to the detected luminescent emissions among the elements of the representative vectors; and
classifying the mapped extracellular microparticles into two or more clusters on the basis of the first scatter diagram.
   [2]

The analysis method according to [1], further including
creating a second scatter diagram by mapping at least a portion of the extracellular microparticles contained in the population of extracellular microparticles in the same manner as the first scatter diagram, and visualizing one of a plurality of the elements corresponding to the detected luminescent emissions among the elements of the representative vectors in each of the mapped extracellular microparticles.
   [3]

The analysis method according to [2], further including:
displaying the first and second scatter diagrams; and
identifying characteristics of at least one of the clusters on the basis of the displayed first and second scatter diagrams.
   [4]

The analysis method according to any one of [1] to [3],
wherein, in creating the first scatter diagram, extracellular microparticles in which all of the elements corresponding to the detected luminescent emissions among the elements of the representative vectors are equal to or less than a threshold are excluded from mapping.
   [5]

The analysis method according to any one of [1] to [4],
wherein the population of extracellular microparticles is a population of extracellular microparticles in which at least three types selected from the group consisting of nucleic acids, proteins, lipids, and sugar chains are stained.
   [6]

The analysis method according to [5],
wherein the population of extracellular microparticles is a population of extracellular microparticles in which all of the nucleic acids, proteins, lipids, and sugar chains are stained.
   [7]

An analysis method for extracellular microparticles, including:
for each extracellular microparticle in a population of extracellular microparticles, generating a feature vector corresponding to the extracellular microparticle by detecting light scattered from incident light and a plurality of luminescent emissions having different peak wavelengths, the luminescent emissions being generated from components of the extracellular microparticle or labeling substances bound to the components in response to the incident light; and
classifying the population of extracellular microparticles into two or more clusters on the basis of the similarity of representative vectors composed of part or all of the elements of the feature vectors.
   [8]

An analysis method for extracellular microparticles, including:
irradiating an extracellular microparticle with incident light, and detecting light scattered from the incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle; and
analyzing characteristics of the extracellular microparticle on the basis of analysis data obtained by analyzing a population of extracellular microparticles by the analysis method according to any one of [1] to [7] and the acquired feature vector.
   [9]

A separation method for extracellular microparticles, including
for an extracellular microparticle in a population of extracellular microparticles, detecting light scattered from incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle;
determining whether the extracellular microparticle has a predetermined characteristic on the basis of analysis data obtained by analyzing a population of extracellular microparticles by the analysis method according to any one of [1] to [7] and the acquired feature vector; and
separating, when determination is made that the extracellular microparticle has the predetermined characteristic, the extracellular microparticle from the population of extracellular microparticles.
   [10]

An analysis device or analysis system for extracellular microparticles, including:
a detection unit configured to detect, for each extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light; and
an information processing unit including
a feature vector generating means for generating a feature vector corresponding to the extracellular microparticle in the population of extracellular microparticles on the basis of the detected scattered light and the plurality of luminescent emissions,
a scatter diagram creating means for creating a first scatter diagram by mapping, in two or three dimensions, at least a portion of the extracellular microparticles contained in the population of extracellular microparticles on the basis of the similarity of representative vectors composed of part or all of the elements of the acquired feature vectors, and labeling each of the mapped extracellular microparticles on the basis of at least a plurality of the elements corresponding to the detected luminescent emissions among the elements of the representative vectors, and
a cluster generating means for generating two or more clusters, to which the extracellular microparticles belong, by assigning cluster information to each of the extracellular microparticles mapped on the first scatter diagram.
   [10-1]

An analysis program for extracellular microparticles that causes an information processing unit in an analysis device for extracellular microparticles to function as:
a feature vector generating means for generating a feature vector corresponding to the extracellular microparticle in the population of extracellular microparticles on the basis of the detected scattered light and the plurality of luminescent emissions,
a scatter diagram creating means for creating a first scatter diagram by mapping, in two or three dimensions, at least a portion of the extracellular microparticles contained in the population of extracellular microparticles on the basis of the similarity of representative vectors composed of part or all of the elements of the acquired feature vectors, and labeling each of the mapped extracellular microparticles on the basis of at least a plurality of the elements corresponding to the detected luminescent emissions among the elements of the representative vectors, and
a cluster generating means for generating two or more clusters to which the extracellular microparticles belong by assigning cluster information to each of the extracellular microparticles mapped on the first scatter diagram.
   [11]

An analysis device for extracellular microparticles, including:
a detection unit configured to irradiate an extracellular microparticle with incident light and detect light scattered from incident light and a plurality of luminescent emissions having different peak wavelengths, the luminescent emissions being generated from components of the extracellular microparticle or labeling substances bound to the components in response to the incident light; and
an information processing unit including
a feature vector generating means for generating a feature vector corresponding to the extracellular microparticle on the basis of the detected scattered light and the plurality of luminescent emissions, and
a characteristic analysis means for analyzing characteristics of the extracellular microparticle on the basis of analysis data obtained by analyzing a population of extracellular microparticles by the analysis method according to any one of [1] to [7] and the acquired feature vector.
   [11-1]

An analysis program for extracellular microparticles that causes an information processing unit in an analysis device for extracellular microparticles to function as:
a feature vector generating means for generating a feature vector corresponding to the extracellular microparticle on the basis of the detected scattered light and the plurality of luminescent emissions, and
a characteristic analysis means for analyzing characteristics of the extracellular microparticle on the basis of analysis data obtained by analyzing a population of extracellular microparticles by the analysis method according to any one of [1] to [7] and the acquired feature vector.
   [12]

A separation device for extracellular microparticles, including:
a detection unit configured to detect, for an extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light;
an information processing unit including
a feature vector generating means for generating a feature vector corresponding to the extracellular microparticle on the basis of the detected scattered light and the plurality of luminescent emissions, and
a determination means for determining whether the extracellular microparticle has a predetermined characteristic on the basis of analysis data obtained by analyzing a population of extracellular microparticles by the analysis method according to any one of [1] to [7] and the acquired feature vector; and
a separation unit configured to separate, when determination is made that the extracellular microparticle has the predetermined characteristic, the extracellular microparticle from the population of extracellular microparticles.
   [12-1]

An analysis program for extracellular microparticles that causes an information processing unit in a separation device for extracellular microparticles to function as:
a feature vector generating means for generating a feature vector corresponding to the extracellular microparticle on the basis of the detected scattered light and the plurality of luminescent emissions, and
a determination means for determining whether the extracellular microparticle has a predetermined characteristic on the basis of analysis data obtained by analyzing a population of extracellular microparticles by the analysis method according to any one of [1] to [7] and the acquired feature vector.
   [13]

A method of preparing a sample containing a population of extracellular microparticles for use in the analysis method according to any one of [1] to [7], the method including
labeling a sample containing a population of extracellular microparticles with a plurality of labeling substances that bind to components of the extracellular microparticles.
   [14]

The method according to [13],
wherein the plurality of labeling substances include at least three types selected from the group consisting of a nucleic acid staining reagent, a protein staining reagent, a lipid staining reagent, and a sugar chain staining reagent.
   [15]
The method according to [13] or [14], wherein the plurality of labeling substances include a compound having a molecular weight of 20,000 or less.

### EXAMPLES

Hereinafter, the present invention will be described in more detail with reference to examples and comparative examples. The present invention is not limited to the following examples.

### 1. Method

### [Setup of BD Influx Cell Sorter]

A BD Influx cell sorter (BD Biosciences) with 355, 405, 488, 561, and 640 nm lasers was placed in a Class II Type All Biosafety Cabinet (Baker). In order to collect the forward scattering signal, an optical system including a high NA, long working distance, 20× objective lens, a pinhole with a diameter of 0.7 mm, and two photomultiplier tubes (hereinafter referred to as “PMT”) that detect light with different polarization directions (FSC-par and FSC-per) was used. When the sensitivity of FSC was adjusted to detect 100 nm particles, 500 nm particles exhibited a saturation signal in FSC, and polarized FSC (FSC-perp) was available for analysis of larger particles (>200 nm). Therefore, a wide range of sizes of extracellular microparticles was analyzed using FSC and FSC-perp. Since it has been reported that an 8 mm obscuration bar was superior to a conventional 5 mm bar in reducing the level of noise signals in FSC (Arkesteijn, G. J. A. et al. Improved Flow Cytometric Light Scatter Detection of Submicron-Sized Particles by Reduction of Optical Background Signals. Cytometry A 97, 610-619 (2020)), an 8 mm aluminum bar was produced on a 2 mm bar.

The nozzle size (70, 86, 100 and 140 μm) did not significantly affect the resolution of extracellular microparticle analysis, but when the flow rate of the sheath liquid increased, the amount of signals from particles decreased. Therefore, a 70 μm nozzle was selected, and the sheath pressure was adjusted to 24.0 p.s.i. Laser alignment was optimized with ultra rainbow fluorescent particles (Spherotech) according to the user guide. All signals were collected by a PMT detector, and displayed as height signals on a logarithmic scale (10⁰ to 10⁴). In order to determine an optimal threshold trigger for extracellular microparticle analysis, the voltage of the FSC signal was adjusted to 33, and 100 nm fluorescent beads (Invitrogen) were measured. An FSC threshold of 0.58 to 0.6 was used. The FSC-perp voltage was set to 33 (for comparison with FSC) or 14 (for analysis of large particles and cells). For extracellular microparticle analysis, the fluorescence voltage was adjusted so that the signal intensity of noise was less than 10¹. Unless otherwise stated, the sample pressure was set between 24.9 and 25.2 p.s.i., and the detection rate was set not to exceed 30,000/second.

### [Sorting of Fluorescent Beads]

100 nm (Invitrogen), and 200, 500, and 1,000 nm (Polysciences) beads were mixed in PBS, and analyzed on a BD Influx cell sorter. In the sorting setup, droplet formation not only impaired the SSC signal but also increased the level of noise signals at an FSC threshold trigger considered to be due to a change in the angle of incidence of laser beams on the liquid surface of the sheath liquid due to droplet formation. It was found that this effect could be reduced by lowering the nozzle position and the break off position for droplets. According to these preliminary findings, in order to obtain the highest break off position, the drop frequency was first adjusted between 60.2 and 60.8. Next, the piezoelectric amplitude was set to the lowest value at which a stable test flow (generally 3.0 to 4.5) was formed. The charging timing was optimized using AccuDrop beads (BD Biosciences). It was found that the optimal charging timing changed as the amount of noise increased, and because the target droplet collection failed at the optimal charging timing with an FSC threshold of 0.6, in order to eliminate noise signals, the charging timing should be optimized under an increased FSC threshold trigger (about 3.0). After the charging timing was adjusted, the FSC threshold trigger was returned to 0.58 to 0.6. Next, target particles were sorted in a bidirectional sorting mode and collected into uncoated 1.5 or 5 mL tubes.

Mixtures of 100, 200, 500, and 1,000 nm beads were measured on a BD Aria III cell sorter and BD LSRFortessa using an original optical system. Detection of 100 to 500 nm particles using BD Aria III or detection of 100 to 200 nm particles using BD LSRFortessa was difficult due to saturated noise signals according to the FSC threshold trigger. The FSC voltage and threshold were adjusted so that the level of noise signals was below 5,000 events/second. For detection of fluorescent beads using CytoFLEX LX (Beckman coulter), a violet SSC threshold was applied according to the small particle analysis user guide. Sorting of 1,000 nm particles using BD Aria III was performed in a purity mode. The sorted fractions were reanalyzed with BD Influx. The purity of the sorted samples was calculated as the ratio of each group to the sum of the 100, 200, 500, and 1,000 nm groups.

### [Preparation of Sample]

C57BL/6J mice (6- to 8-week old) were purchased from CLEA, Japan. Mouse serum was collected by centrifugation of blood at 2,000×g for 10 minutes and kept at −80 °C until use. For analysis of mouse BALF, mice were infected intranasally with 0.1 to 10 LD₅₀ of A/Puerto Rico/8/1934 influenza (H1N1) virus in 30 μL of PBS under anesthesia.

For a preventive treatment, mice were inoculated with 1 μg of monovalent influenza split vaccine (SV) containing HA from New Caledonia/20/1999 (H1N1) (Research Institute for Microbial Diseases, Osaka University) with or without 300 ng of aluminum hydroxide salt (Alum) (Invivogen) or 10 μg of K3 CpG oligodeoxynucleotide (GeneDesign) at the base of the tail 17 days and 7 days before virus infection.

For a curative treatment, mice were orally administered 20 mg/kg/day of oseltamivir phosphate (selleck) on the 1^{st} to 3^{rd} days (dpi). On the 4^{th} day, the lungs were washed with PBS (Natalai tesque) to collect BALF. A 22-gauge plastic cannula (Terumo) was inserted into the incised trachea. Next, 0.7 mL of PBS filled in a 1 mL syringe was injected and aspirated. During first washing, about 0.5 mL of BALF was collected (the dead volume was about 0.2 mL). Next, washing was performed again with 0.5 mL of PBS, and 0.5 mL of BALF (a total of 1 mL) was collected. Next, in order to remove large aggregates, BALF was filtered through a 70 μm cell strainer, and used in the following assay.

After obtaining informed consent, human heparin-treated blood was collected from healthy adults. In order to obtain human peripheral blood mononuclear cells (hPBMCs) and plasma, blood was diluted with an equal volume of PBS, and gently layered on the top of Ficoll Histopaque (GE healthcare). After centrifugation at 900×g for 20 minutes, the hPBMC-containing white buffy coat and the upper layer plasma were collected. The plasma was kept at −80 °C until use. PBMCs were washed twice with a medium, and used in the subsequent experiment as described below.

### [Multiparametric Analysis of Extracellular Microparticles and Subsequent Data Processing]

For multiparametric analysis of extracellular microparticles, BALF was centrifuged at 400×g for 5 minutes to remove cells, and the supernatant was collected for subsequent staining. When both the extracellular microparticles and cells were analysis targets, BAL cells were centrifuged at 400×g for 5 minutes and concentrated 30-fold. Next, the samples were diluted 5-fold with an Annexin V staining buffer solution. Because the presence of other staining reagents prevented staining of membranous particles with CellMask deep red, CellMask deep red was mixed with the sample first, prior to the other staining reagents. Then, the other staining reagents were added at the following final concentrations: CellMask deep red ( 1/10000), SYBR gold ( 1/5000), Hoechst33258 ( 1/100), Live/Dead stain Near-IR (Invitrogen, 1/1000), BV510 Annexin V ( 1/20), CF405M WGA ( 1/200), CF568 PNA ( 1/20), RPE-CF647T anti-HA antibody ( 1/100), PE-CF594 anti-siglec-F antibody (BD bioscicence, 1/100), PE-Cy7 anti-CD11c antibody (Biolegend, 1/100), and BV785 anti-Ly6C antibody (Biolegend, 1/100). After incubation for 20 minutes, the samples were diluted with an Annexin V staining buffer solution, and measured on a BD Influx cell sorter.

Data was analyzed in FlowJo (v10.7.2) according to the following procedure. First, correction of the fluorescence signal was performed manually by single-staining control. The correction value was determined as the minimum percentage necessary to avoid detection of stained particles in other channels. Next, all particles or stained particles extracted by applying an “or” gate were analyzed by t-SNE (opt-SNE, iterations: 1000, perplexity: 30), UMAP, or FlowSOM. All fluorescence parameters for staining extracellular microparticles were included in the t-SNE analysis. When cells were analyzed by t-SNE, cell surface markers were included only as parameters. The results of t-SNE analysis were visualized as dot plots or counter plots with colors indicated in the order of priority shown in each drawing.

### [Feature Engineering of Flow Cytometry Data for Machine Learning]

In order to use flow cytometry analysis data of individual BALF for machine learning, features were generated from flow cytometry analysis raw data by binning. Specifically, the particles detected by flow cytometry had multidimensional information composed of the scattered light and fluorescence signals. Therefore, in order to obtain the extracellular microparticle concentration in BALF as a feature while retaining as much information as possible, seven bins were created, and the particles were divided into seven sizes based on the FSC signal. In addition, the particles with each size were additionally divided into two bins for each fluorescence intensity. By repeating this process for all fluorescence signals, BALF particles were classified into 3,584 bins. These procedures were performed in FlowJo. After blank bins were removed, count data of 2,000 bins was used as characteristics of the BALF samples.

### [Machine Learning Analysis]

The machine learning framework was built on Python (version 3.10) using libraries (scikit-learn (version 1.0.2), numpy (version 1.21.5), and pandas (version 1.4.2)). Each predictor was built as an ensemble of three scikit-learn pipelines. In other words, the response prediction was calculated as the average of scores generated by the three classification pipelines.

### [Statistical Analysis]

Experiments were performed independently at least twice. The experimental results were shown as average±SEM. The statistical significance of differences between groups was determined by the unpaired Student's t-test or one-way ANOVA using Tukey's multiple comparison test. The significant differences were annotated with asterisks: P<0.05: *, P<0.01: **, P<0.001: ***.

### 2. Optimization of Optical System for Detection and Classification of Extracellular Microparticles

One main problem in flow cytometry analysis of extracellular microparticles was optimizing the threshold trigger to eliminate noise signals. Lowering the threshold was necessary to detect smaller particles, but it also led to an increase in the level of noise signals and signal process overload, and as a result, the number of detected target particles decreased. Fluorescence-based thresholding is an effective method for separating fluorescently labeled particles from noise signals, but is not suitable for comprehensive analysis because it eliminates non-labeled particles. Therefore, in order to analyze all particles in the sample, optimization of the optical system for FSC-based thresholding was attempted.

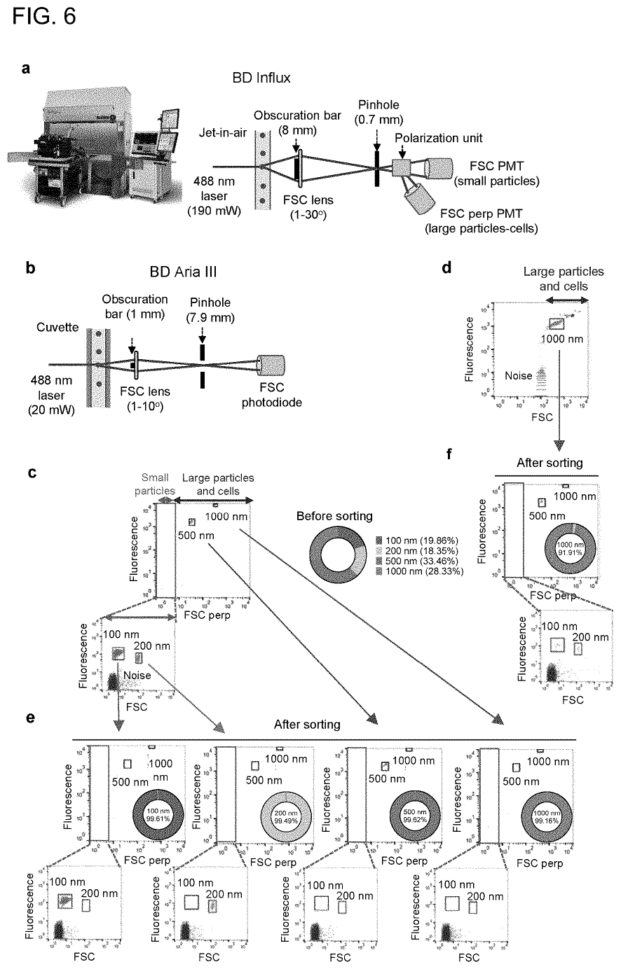

The BD Influx cell sorter was selected for the analysis and sorting of extracellular microparticles because a wide range of FSC could be collected with a highly sensitive PMT detector and multiple FSC parameters of a polarizing unit. According to the previous documents (Arkesteijn, G. J. A. et al. Improved Flow Cytometric Light Scatter Detection of Submicron-Sized Particles by Reduction of Optical Background Signals. Cytometry A 97, 610-619 (2020); and Nolte-'t Hoen, E. N. et al. Quantitative and qualitative flow cytometric analysis of nanosized cell-derived membrane vesicles. Nanomedicine 8, 712-720 (2012)), in order to detect 100 nm fluorescent beads, an 8 mm obscuration bar was used. In addition, the polarizing unit was used to cover a wide range of sizes (FIG. 6a). Since FSC thresholding exhibited better results in detection of 100 nm beads than polarized FSC (FSC-perp), small (100 to 200 nm) particles and large (>200 nm) particles were analyzed by general FSC and polarized FSC, respectively (FIG. 6c).

Next, the particle sorting test was performed using the BD Influx cell sorter. According to FSC thresholding with an optimized optical system, the fluorescent beads with high purity (>99%) and different sizes could be separated (FIG. 6e). In BD Aria III, which is a conventional cell sorter including a photodiode FSC, it was difficult to detect and sort beads with a size of less than 1,000 nm due to saturation of noise signals (FIGS. 6b and 6d). By reanalyzing on a BD Influx cell sorter, 1,000 nm particle fractions sorted on BD Aria III were found to be significantly contaminated with other beads. This is thought to be due to the fact that non-optimized BD Aria III was unable to eliminate droplets containing both 1,000 nm and 100 to 500 nm particles (FIG. 6f). In addition, BD LSRFortessa and CytoFLEX analyzers including a photodiode FSC had a lower resolution of the FSC signal than the BD Influx.

In order to further verify the size range of dual FSC signals on the BD Influx cell sorter, BALF was measured as a sample containing both cells and extracellular microparticles. By changing the FSC-perp and SSC scales from log to linear, cells were detected in the same manner as in typical cytometric analysis (FIG. 7a). In addition, the extracellular microparticles were shown as a continuous group ranging from the FSC to FSC-perp scale. This is thought to reflect the non-uniform size distribution of extracellular microparticles (FIG. 7b).

### 3. Evaluation of High-Resolution Characteristic of Extracellular Microparticles Based on Multicolor Staining

In order to evaluate the potential of extracellular microparticle staining in order to identify groups in non-uniform extracellular microparticles, eight-color staining of BALF containing extracellular microparticles was performed by targeting nucleic acids, lipid membranes, sugar chains and proteins without a washing step (FIG. 7a). In addition to the above fluorescence probe, Live/Dead fixable dead cell stain, and amine-reactive fluorescent staining reagents were used as protein labeling substances. These were used to identify non-specific binding of antibodies to large extracellular microparticles. In addition, in order to detect virus particles, fluorescently labeled anti-hemagglutinin (HA) antibodies were prepared. As a result, small extracellular microparticles in the FSC region were stained using each probe, and some of these particles were significantly increased by virus infection (FIG. 7c). In order to visualize non-uniformity of extracellular microparticles in BALF, T-distributed Stochastic Neighbor Embedding (t-SNE) analysis was performed based on the fluorescence and FSC signal, and it was found that probe-stained particles appeared as a group separated from unstained particles (FIG. 7d). Because unstained particles including mainly noise signals appeared as the main group, and the resolution of the stained group decreased, an “or” gate was then set to perform t-SNE analysis using only particles stained using at least one fluorescence probe (FIG. 7e). According to this data processing, the resolution of each group increased, and virus particles, particles containing sugar chains or nucleic acids, and other membranous particles could be presented as separate groups (FIG. 7f). In addition, as another dimension reduction algorithm, uniform manifold approximation and projection (UMAP) analysis was performed. According to the UMAP analysis, SYBR gold-positive particles were shown as separated groups, which suggested their unique characteristics. In addition, using a self-organizing map (FlowSOM), the SYBR gold-positive particle group was clustered as a single group.

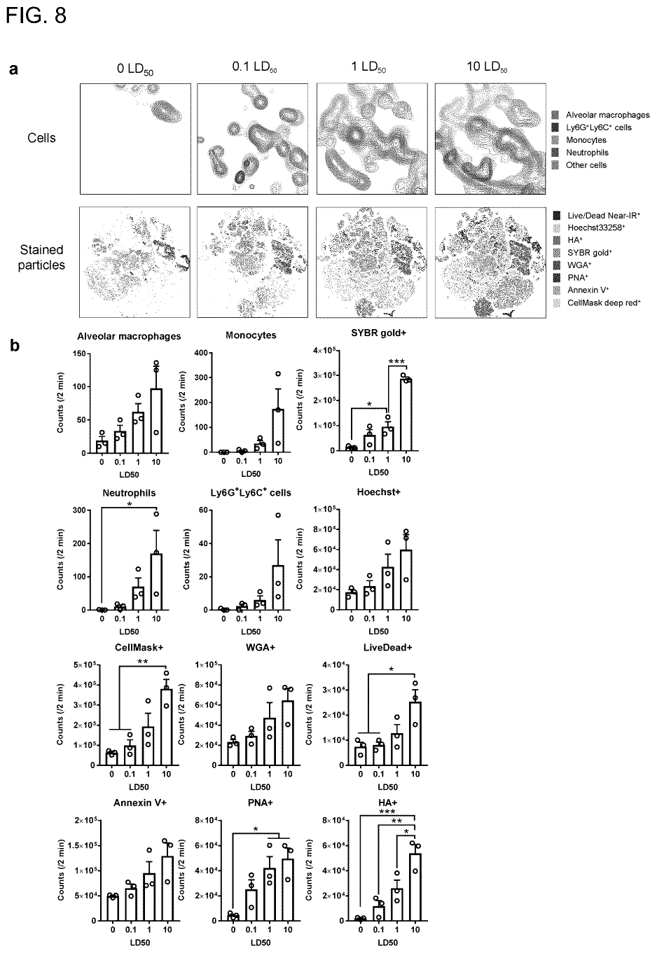

In order to further verify the utility of this multiparametric analysis, this workflow was applied to evaluation of the severity of virus infection. In this experiment, cells as well as extracellular microparticles were stained by antibodies targeting surface markers, and analyzed using a conventional gating strategy and subsequent machine learning. As a result, the number of inflammatory cells including neutrophils and monocytes increased in response to the influenza virus dose (FIGS. 8a and 8b). According to analysis of stained extracellular microparticles, it was suggested that the number of some groups such as SYBR gold-positive particles correlated with the severity of diseases (FIGS. 8a and 8b). In addition, in other experiments using prevention and treatment models, it was suggested that sugar chain-positive particles and HA-positive particles could reflect the state during virus infection. In addition, when extracellular microparticles in the medium were stained, it was confirmed that extracellular microparticle staining could be applied to other liquids such as a PBMC culture supernatant.

### 4. Selective Separation of Extracellular Microparticles

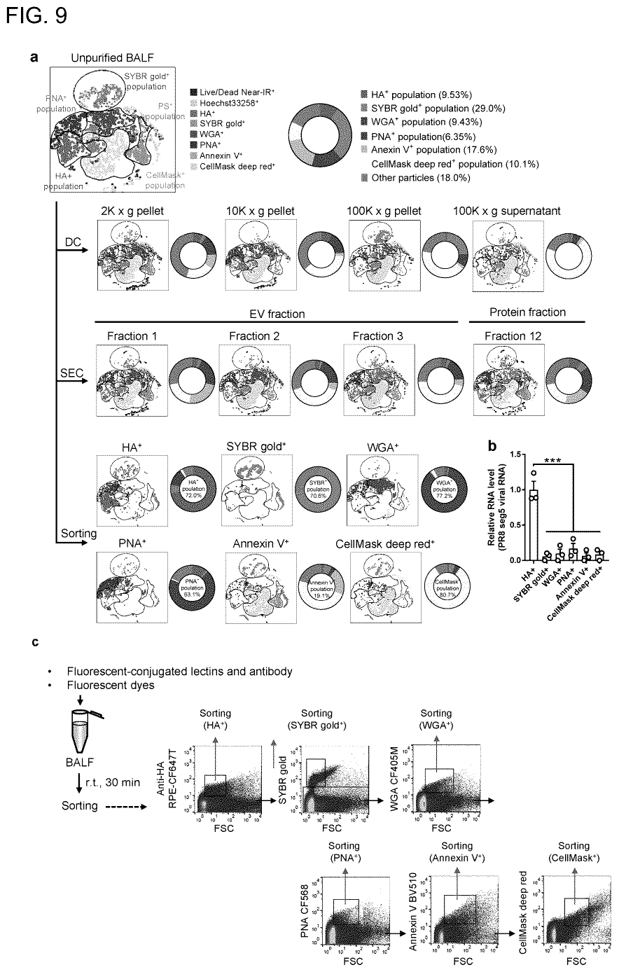

Next, it was evaluated whether the population of extracellular microparticles indicated by t-SNE could be separated by flow cytometric sorting. According to the gating strategy shown in FIG. 9c, the main populations of extracellular microparticles were sorted. For reanalysis of the sorted SYBR gold-positive particles, it was necessary to re-stain the solution after sorting with SYBR gold. This was thought to be probably caused by dissociation of the staining reagent from the sorted nucleic acids, but some larger particles in the sheath liquid were also stained. Clustering analysis gated on target extracellular microparticles showed concentration of each group (FIG. 9a). When the population of extracellular microparticles was gated on the t-SNE plot and the ratio thereof was calculated, the purity of the target group was lower than the purity when the fluorescent beads were sorted (FIGS. 6e and 9a). This is particularly noticeable for Annexin V in which calcium ions were necessary to maintain binding, but was thought to be caused by dissociation of the fluorescence probe as with SYBR gold. In addition, for comparing a conventional separation method for extracellular microparticles, when stained particles in fractions collected by a differential centrifugation method (DC) and size exclusion chromatography (SEC) were analyzed, the separation ability was found to be lower than that of sorting by flow cytometry (FIG. 9a). The removal of SYBR gold-positive particles from the extracellular microparticle fractions after SEC separation supported the hypothesis that, in an analysis method requiring a washing step, not only the unbound staining reagent but also particles themselves were washed away, preventing comprehensive analysis of the extracellular microparticles. RT-qPCR of viral RNA in the sorted fractions showed that viral RNA was concentrated only in HA-positive fractions and not concentrated in other groups. This suggested that virus particles were successfully separated by flow cytometric sorting (FIG. 9b).

### 5. Identification of Population of Non-Membrane Extracellular Microparticles Based on Multi-Color Analysis and Sorting

It was attempted to clarify the details of extracellular nucleic acids including SYBR gold-positive particles from among extracellular microparticles classified by multiparametric analysis. Most nucleic acid-containing particles in BALF were divided into an SYBR gold and Hoechst33258 positive group and an SYBR gold-positive group. In addition, SYBR gold-positive particles were divided into a small group and a large group based on the FSC signal. Sorting and subsequent PCR analysis of these groups revealed a distinctive feature of nucleic acids in the large SYBR gold-positive particles, namely that they contained a higher amount of mitochondrial DNA (mtDNA) compared to other groups. To further characterize the most abundant nucleic acid positive group, small SYBR gold-positive particles, it was confirmed that the small SYBR gold-positive particles were negative for CellMask deep red. Next, it was examined whether these nucleic acids formed complexes with proteins such as histone. Co-staining of SYBR gold-positive particles with anti-citrullinated histone H3(cit-H3) antibodies suggested that small SYBR gold-positive particles were DNA/histone complexes fragmented from neutrophil extracellular traps (NETs). Based on the results of flow cytometry in which most cit-H3 positive particles corresponded to small SYBR gold-positive particles, cit-H3 positive particles in BALF were analyzed under a transmission electron microscope (TEM), and thus the structure of small SYBR gold-positive particles was evaluated. As a result, cit-H3 positive particles were observed as a non-membrane type, which was similar to the TEM image of chromatin. These results suggested that the strategy based on the present invention for the analysis and sorting of extracellular microparticles could clarify characteristics of not only membrane particles but also non-membrane particles. Next, it was evaluated whether these DNA/histone complexes could be analyzed using other flow cytometers. nanoFCM, which is specialized for nanoparticle analysis, also detected SYBR gold-positive cit-H3 positive particles. The estimated size of these particles determined by nanoFCM was about 70 nm, suggesting that the detection limit of the BD Influx cell sorter was less than 100 nm.

### 6. Conclusion

As described above, in order to achieve staining of the total extracellular microparticles, by applying the concept of staining a plurality of components of cells using various fluorescence probes for detailed classification of cells to extracellular microparticle analysis, a robust workflow for analysis and separation of extracellular microparticles was developed. When the cell sorter was finely adjusted, it became possible to detect and sort particles with non-uniform sizes ranging from 100 nm to 1 μm or more. As a result of fluorescence probe screening for lipids, nucleic acids, sugar chains and proteins, an appropriate combination of staining reagents for multicolor staining of extracellular microparticles without the need of a washing step was clarified. In addition, high-purity fractionation of target particles from bulk samples, which could not be achieved by a conventional isolation method, was also confirmed. The present invention proposes, for example, a universal method of analyzing and sorting all particles in a biological sample through a simple procedure.

### 7. Changing Cell Sorter

It was confirmed that, even if other cell sorters were used in place of the BD Influx cell sorter, the extracellular microparticles could be analyzed as described in the above 1 to 6. As the cell sorter, BD FACSDiscover S8 (Becton, Dickinson and Company) was used. The setup was the same as for the BD Influx cell sorter except that the SSC threshold trigger was used in place of the FSC threshold trigger.

### [Sorting of Fluorescent Beads]

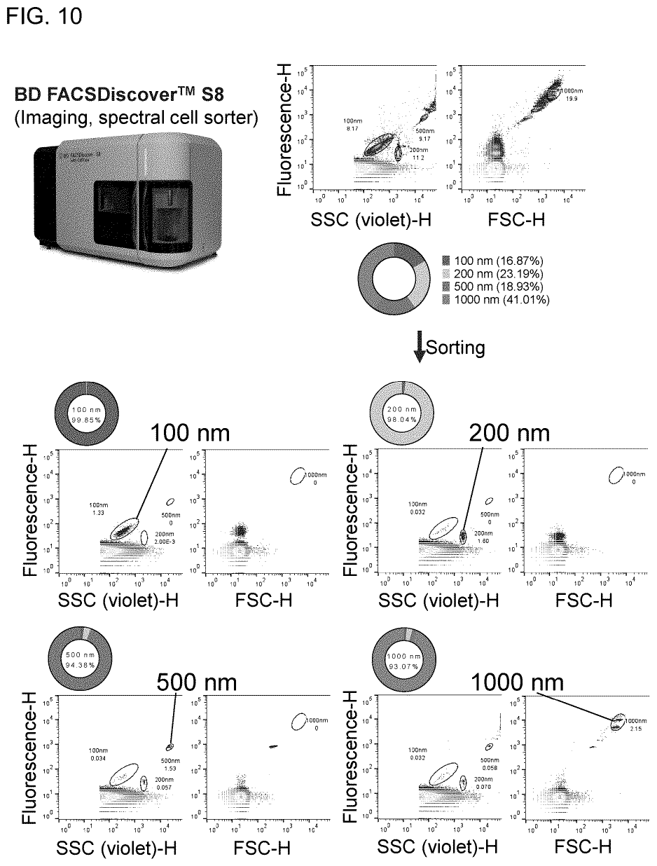

Sorting of fluorescent beads was performed using the method described in the above 1. It was confirmed that the fluorescent beads with high purity and different sizes could be separated according to SSC thresholding (FIG. 10).

### [Evaluation of High-Resolution Characteristics of Extracellular Microparticles]

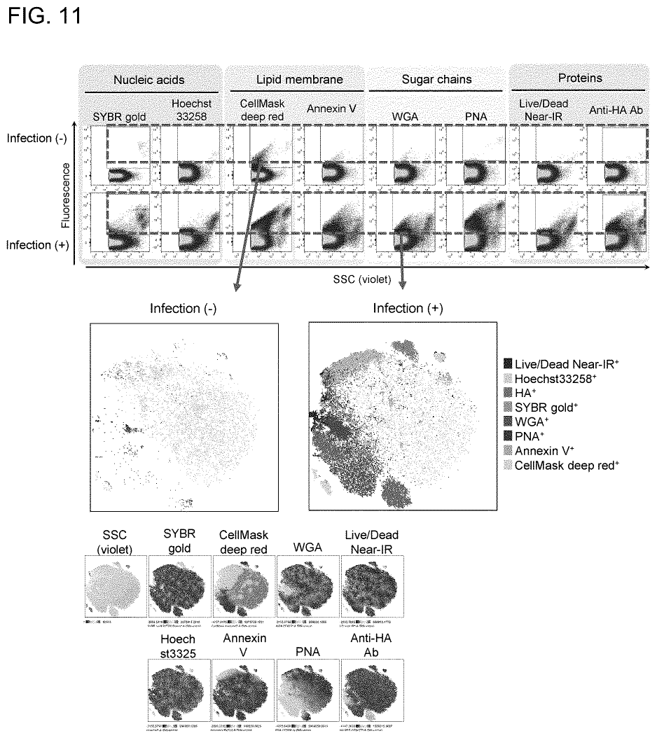

Using the method described in the above 1 and 3, high-resolution characteristics of extracellular microparticles were evaluated. As measurement samples, BALFs from mice intranasally infected with influenza virus and from uninfected mice were used. The results are shown in FIG. 11. FIG. 11 shows that the proportion of extracellular microparticles classified into predetermined types increased in mice intranasally infected with influenza virus.

### [Selective Separation of Extracellular Microparticles]

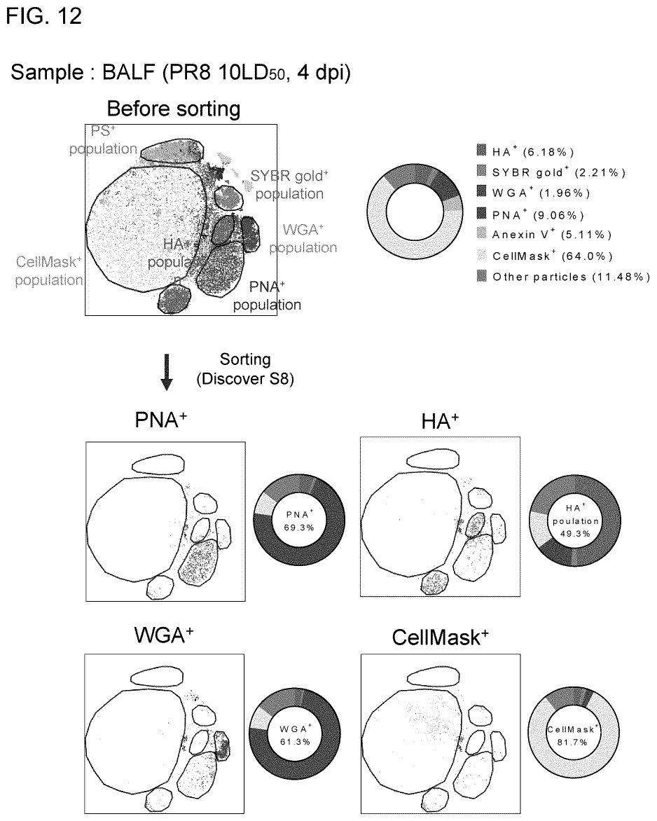

Using the method described in the above 1 and 4, the population of extracellular microparticles indicated by t-SNE was separated by flow cytometric sorting. It was found that specific extracellular microparticles could be selectively separated in the same manner as when the BD Influx cell sorter was used (FIG. 12).

### REFERENCE SIGNS LIST

1 Analysis device
2 Detection unit
3 Information processing unit
4 Input unit
5 Output unit
31 RAM
32 ROM
33 Storage
34 CPU
35 Receiving means
36 Transmitting means
37 System bus

1. An analysis method for extracellular microparticles, comprising:
for each extracellular microparticle in a population of extracellular microparticles, generating a feature vector corresponding to the extracellular microparticle by detecting light scattered from incident light and a plurality of luminescent emissions having different peak wavelengths, the luminescent emissions being generated from components of the extracellular microparticle or labeling substances bound to the components in response to the incident light;
creating a first scatter diagram by mapping, in two or three dimensions, at least a portion of the extracellular microparticles contained in the population of extracellular microparticles on the basis of the similarity of representative vectors composed of part or all of the elements of the feature vectors, and labeling each of the mapped extracellular microparticles on the basis of at least a plurality of the elements corresponding to the detected luminescent emissions among the elements of the representative vectors; and
classifying the mapped extracellular microparticles into two or more clusters on the basis of the first scatter diagram.

2. The analysis method according to claim 1, further comprising
creating a second scatter diagram by mapping at least a portion of the extracellular microparticles contained in the population of extracellular microparticles in the same manner as the first scatter diagram, and visualizing one of a plurality of the elements corresponding to the detected luminescent emissions among the elements of the representative vectors in each of the mapped extracellular microparticles.

3. The analysis method according to claim 2, further comprising:
displaying the first and second scatter diagrams; and
identifying characteristics of at least one of the clusters on the basis of the displayed first and second scatter diagrams.

4. The analysis method according to claim 1,
wherein, in creating the first scatter diagram, extracellular microparticles in which all of the elements corresponding to the detected luminescent emissions among the elements of the representative vectors are equal to or less than a threshold are excluded from mapping.

5. The analysis method according to claim 1,
wherein the population of extracellular microparticles is a population of extracellular microparticles in which at least three types selected from the group consisting of nucleic acids, proteins, lipids, and sugar chains are stained.

6. The analysis method according to claim 5,
wherein the population of extracellular microparticles is a population of extracellular microparticles in which all of the nucleic acids, proteins, lipids, and sugar chains are stained.

7. An analysis method for extracellular microparticles, comprising:
for each extracellular microparticle in a population of extracellular microparticles, generating a feature vector corresponding to the extracellular microparticle by detecting light scattered from incident light and a plurality of luminescent emissions having different peak wavelengths, the luminescent emissions being generated from components of the extracellular microparticle or labeling substances bound to the components in response to the incident light; and
classifying the population of extracellular microparticles into two or more clusters on the basis of the similarity of representative vectors composed of part or all of the elements of the feature vectors.

8. An analysis method for extracellular microparticles, comprising:
irradiating an extracellular microparticle with incident light, and detecting light scattered from the incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle; and
analyzing characteristics of the extracellular microparticle on the basis of analysis data obtained by analyzing a population of extracellular microparticles by the analysis method according to claim 1 and the acquired feature vector.

9. A separation method for extracellular microparticles, comprising:
for an extracellular microparticle in a population of extracellular microparticles, detecting light scattered from incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle;
determining whether the extracellular microparticle has a predetermined characteristic on the basis of analysis data obtained by analyzing a population of extracellular microparticles by the analysis method according to claim 1 and the acquired feature vector; and
separating, when determination is made that the extracellular microparticle has the predetermined characteristic, the extracellular microparticle from the population of extracellular microparticles.

10. An analysis device for extracellular microparticles, comprising:
a detection unit configured to detect, for each extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light; and
an information processing unit comprising:
a feature vector generating means for generating a feature vector corresponding to the extracellular microparticle in the population of extracellular microparticles on the basis of the detected scattered light and the plurality of luminescent emissions,
a scatter diagram creating means for creating a first scatter diagram by mapping, in two or three dimensions, at least a portion of the extracellular microparticles contained in the population of extracellular microparticles on the basis of the similarity of representative vectors composed of part or all of the elements of the acquired feature vectors, and labeling each of the mapped extracellular microparticles on the basis of at least a plurality of the elements corresponding to the detected luminescent emissions among the elements of the representative vectors, and
a cluster generating means for generating two or more clusters, to which the extracellular microparticles belong, by assigning cluster information to each of the extracellular microparticles mapped on the first scatter diagram.

11. An analysis device for extracellular microparticles, comprising:
a detection unit configured to irradiate an extracellular microparticle with incident light and detect light scattered from incident light and a plurality of luminescent emissions having different peak wavelengths, the luminescent emissions being generated from components of the extracellular microparticle or labeling substances bound to the components in response to the incident light; and
an information processing unit comprising:
a feature vector generating means for generating a feature vector corresponding to the extracellular microparticle on the basis of the detected scattered light and the plurality of luminescent emissions, and
a characteristic analysis means for analyzing characteristics of the extracellular microparticle on the basis of analysis data obtained by analyzing a population of extracellular microparticles by the analysis method according to claim 1 and the acquired feature vector.

12. A separation device for extracellular microparticles, comprising:
a detection unit configured to detect, for an extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light;
an information processing unit comprising:
a feature vector generating means for generating a feature vector corresponding to the extracellular microparticle on the basis of the detected scattered light and the plurality of luminescent emissions, and
a determination means for determining whether the extracellular microparticle has a predetermined characteristic on the basis of analysis data obtained by analyzing a population of extracellular microparticles by the analysis method according to claim 1 and the acquired feature vector; and
a separation unit configured to separate, when determination is made that the extracellular microparticle has the predetermined characteristic, the extracellular microparticle from the population of extracellular microparticles.

13. A method of preparing a sample comprising a population of extracellular microparticles for use in the analysis method according to claim 1, the method comprising
labeling a sample comprising a population of extracellular microparticles with a plurality of labeling substances that bind to components of the extracellular microparticles.

14. The method according to claim 13, wherein the plurality of labeling substances comprise at least three types selected from the group consisting of a nucleic acid staining reagent, a protein staining reagent, a lipid staining reagent, and a sugar chain staining reagent.

15. The method according to claim 13, wherein the plurality of labeling substances comprise a compound having a molecular weight of 20,000 or less.

16. An analysis method for extracellular microparticles, comprising:
irradiating an extracellular microparticle with incident light, and detecting light scattered from the incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle; and
analyzing characteristics of the extracellular microparticle on the basis of analysis data obtained by analyzing a population of extracellular microparticles by the analysis method according to claim 7 and the acquired feature vector.

17. A separation method for extracellular microparticles, comprising:
for an extracellular microparticle in a population of extracellular microparticles, detecting light scattered from incident light and a plurality of luminescent emissions having different peak wavelengths, the luminescent emissions being generated from components of the extracellular microparticle or labeling substances bound to the components in response to the incident light, and thus acquiring a feature vector corresponding to the extracellular microparticle;
determining whether the extracellular microparticle has a predetermined characteristic on the basis of analysis data obtained by analyzing a population of extracellular microparticles by the analysis method according to claim 7 and the acquired feature vector; and
separating, when determination is made that the extracellular microparticle has the predetermined characteristic, the extracellular microparticle from the population of extracellular microparticles.

18. An analysis device for extracellular microparticles, comprising:
a detection unit configured to irradiate an extracellular microparticle with incident light and detect light scattered from incident light and a plurality of luminescent emissions having different peak wavelengths, the luminescent emissions being generated from components of the extracellular microparticle or labeling substances bound to the components in response to the incident light; and
an information processing unit comprising:
a feature vector generating means for generating a feature vector corresponding to the extracellular microparticle on the basis of the detected scattered light and the plurality of luminescent emissions, and
a characteristic analysis means for analyzing characteristics of the extracellular microparticle on the basis of analysis data obtained by analyzing a population of extracellular microparticles by the analysis method according to claim 7 and the acquired feature vector.

19. A separation device for extracellular microparticles, comprising:
a detection unit configured to detect, for an extracellular microparticle in a population of extracellular microparticles, light scattered from incident light and a plurality of luminescent emissions having different peak wavelengths, the luminescent emissions being generated from components of the extracellular microparticle or labeling substances bound to the components in response to the incident light;
an information processing unit comprising:
a feature vector generating means for generating a feature vector corresponding to the extracellular microparticle on the basis of the detected scattered light and the plurality of luminescent emissions, and
a determination means for determining whether the extracellular microparticle has a predetermined characteristic on the basis of analysis data obtained by analyzing a population of extracellular microparticles by the analysis method according to claim 7 and the acquired feature vector; and
a separation unit configured to separate, when determination is made that the extracellular microparticle has the predetermined characteristic, the extracellular microparticle from the population of extracellular microparticles.
